(19) United States
(12) Patent Application Publication
(10) Pub. No.: US 2009/0208934 A1
(43) Pub. Date: Aug. 20, 2009

(54) PECAM-1 GENOTYPE

(75) Inventors: Subroto Chatterjee, Columbia, MD (US); Heiming Wei, Singapore (SG)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 11/794,871

(22) PCT Filed: Jan. 5, 2006
(86) PCT No.: PCT/US2006/000525

§ 371 Date: Feb. 19, 2009

Related U.S. Application Data
(60) Provisional application No. 60/641,595, filed on Jan. 5, 2005.

(51) Int. Cl. C12Q 1/68; C40B 40/08; C12N 5/08

(52) U.S. Cl. 435/6; 506/17; 435/366

(57) ABSTRACT

The invention relates to methods of identifying inter-patient differences in genotype of PECAM-1 to diagnose and assess risk of arterial disease. It further relates to methods of identifying therapeutics agents for to treat coronary arterial disease, and to methods for determining and exploiting such differences to improve medical outcomes.

### RELATED APPLICATIONS

This application claims priority from U.S. Provisional Application No. 60/641,595, filed Jan. 5, 2005, entitled “Use of Platelet Endothelial Cell Adhesion Molecule-1 (PECAM-1) Gene Polymorphism in Coronary Artery Disease,” filed Jan. 5, 2005, which is hereby incorporated by reference in its entirety.

### BACKGROUND

Artherosclerosis a chronic inflammatory process initiated by vascular injury induced by atherogenic factors like oxidized low-density lipoprotein (oxLDL), diabetes, and infection¹, and it is characterized by the recruitment of circulating leukocytes to inflamed vascular wall. The latter is predominantly mediated by a group of cellular adhesion molecules (CAMs) expressed on the cell surface, such as selectins, intracellular cell adhesion molecule-1 (ICAM-1), vascular cell adhesion molecule-1 (VCAM-1) and platelet endothelial cell adhesion molecule-1 (PECAM-1, CD 31)²⁻⁴ PECAM-1, a 130-kDa membrane glycoprotein and a member of immunoglobulin (Ig) superfamily, is expressed on the surface of monocytes, some T-lymphocyte subsets, platelets and endothelial cells^{5,6,7} where it concentrates at cell-cell borders. As a trans-membrane glycoprotein, PECAM-1 has 6 Ig-like (homology) extracellular domains (encoded by exon 3 to 8), a short trans-membrane domain (encoded by exon 9) and a short cytoplasmic tail (encoded by exon 10-16)^{8,9}. The importance of the first domain of PECAM-1 (encoded by exon 3) is underscored since PECAM-1 forms homophilic binding via its first or the first plus the second extracellular Ig-like domains or heterophilic binding with other molecules to mediate cell-cell adhesion^{10,11}. It has been suggested that PECAM-1 is a multifunctional cell adhesion molecule involved in angiogenesis¹², intergrin regulation¹³, apoptosis¹⁴ and more importantly, trans-endothelial migration of monocytes (TEM)^{10,15}, Also, PECAM-1 plays a role in plaque formation and thrombosis^{6,16}.

In the United States and most other Western countries, atherosclerosis is the leading cause of illness and death. In the United States alone, it caused almost 1 million deaths in 1992—twice as many as from cancer and 10 times as many as from accidents. Despite significant medical advances, coronary artery disease (which results from artherosclerosis and causes heart attacks) and artherosclerotic stroke are responsible for more deaths than all other causes combined.

Consequently, there is a need in the art to find diagnostic methods, treatments and method of screening for new treatments for artherosclerotic disease. Thus, it would be desirable to have additional methods of treating conditions or diseases affected by PECAM variants to treat or prevent artherosclerotic disease.

### SUMMARY

Provided herein are methods of identifying inter-patient differences in genotype of PECAM-1 to diagnose and assess risk of arterial disease. It further relates to methods of identifying therapeutics agents for treating coronary arterial disease, and to methods for determining and exploiting such differences to improve medical outcomes.

According to one aspect, methods of assessing risk of artherosclerotic disease in a subject comprise determining a PECAM-1 genotype status of a subject, and correlating the genotype status to a subject's risk of developing artherosclerotic disease.

In one embodiment, the method further comprises correlating the genotype status to a therapeutic treatment.

In one embodiment, the genotype status is determined by one or more of immunological methods or sequencing methods.

In another embodiment, the PECAM-1 genotype status at one or more of amino acid positions 125 or 563 are determined.

In a further embodiment, the PECAM-1 genotype status at one or more nucleotide positions 373 or 1688 are determined.

In one embodiment, the PECAM-1 genotype status is determined by PCR methods, immunological methods, sequencing methods, expression level of PECAM-1, level of soluble PECAM-1, enzyme kinetics of PECAM-1, SNP Chip technology, RFLP, gene function assays (such as adhesion, trans-endothelial migration and angiogenesis).

In one embodiment, PCR methods are one or more of real-time PCR, PCR, reverse transcriptase PCR, or allele-specific PCR.

In one embodiment, a variant genotype status or heterozygous genotype status correlates with increased risk of developing an artherosclerotic disease.

In another embodiment, the variant genotype status is one or more of Val at amino acid position 432, G at nucleotide position 373, Asn at amino acid position 563, or A at nucleotide position 1688.

In another embodiment, a wildtype genotype status correlates with decreased risk of developing an artherosclerotic disease.

In a further embodiment, the wildtype genotype status is one or more of Leu at amino acid position 125, C at nucleotide position 373, Ser at amino acid position 563, or G at nucleotide position 1688.

In one embodiment, a homozygous variant PECAM-1 genotype status correlates with unresponsiveness of a tumor to therapeutic treatment with a PECAM-1 genotype status correlating with increased risk of developing an artherosclerotic disease.

In a further embodiment, the homozygous variant PECAM-1 genotype status is one or more of Val at both amino acid positions 125; G at both nucleotide positions 373; Asn at both amino acid positions 563; A at both nucleotide positions 1688; Val at both amino acid positions 125 and Asn at both amino acid positions 563; or G at both nucleotide positions 373 and A at both nucleotide positions 1688.

In one embodiment, the method further comprises administering a therapeutic amount of an anti-artherosclerotic agent to the subject.

In one embodiment, the anti-artherosclerotic agent comprises one or more of statins, fibrate (Clofibrate, Gemfibrozil (e.g. Lopid®), Fenofibrate Bezafibrate (e.g. Bezalip®), Ciprofibrate (e.g. Modalim®))), aspirin, warfarin, niacin, beta-blockers (acebutolol, atenolol, betaxolol, bisoprolol, esmolol, metoprolol, nebivolol, butoxamine, nadolol, oxprenolol, propranolol, pindolol, sotalol, timolol), calcium channel-blockers, angiotensin-converting enzyme (ACE) inhibitors (e.g., Sulfhydryl-containing ACE inhibitors, e.g., Captopril (Capoten®); Dicarboxylate-containing ACE inhibitors, e.g., Enalapril (Vasotec®/Renitec®), Ramipril (Altace®/Tritace®/Ramace®), Quinapril (Accupril®), Perindopril (Coversyl®), Lisinopril (Lisodur®/Prinivil®/Zestril®); phosphonate-containing ACE inhibitors, e.g., Fosinopril (Monopril®); naturally occurring, e.g., casokinins and lactokinins)), angiotension II receptor blockers, vasodilators, cardiac glycosides/anti-arrhythmics, diuretics (spironolactone, amiloride, triamterene, water, cranberry juice, caffeine, acetazolamide, dorzolamide, furosemide, bumetanide, ethacrynic acid, hydrochlorothiazide, bendroflumethiazide, mannitol, and glucose), cholesterol-lowering drugs (Ezetimibe (Zetia®, Ezemibe®, Ezetrol®)), statins (e.g., atorvastatin (Lipitor®), fluvastatin (Lescol®), lovastatin (Mevacor®, Altocor®), not marketed in the UK), pravastatin (Pravachol®, Selektine®, Lipostat®), rosuvastatin (Crestor®), simvastatin (Zocor®, Lipex®), cerivastatin (Lipobay®, Baycol®)—marketing discontinued in 2001 by the manufacturer (Bayer) due to serious side-effects, especially when used in combination with fibrates, and combination of ezetimibe and simvastatin (Vytorin®)), folic acid, PPAR inhibitors, cholesterol efflux agents.

In one embodiment, the method further comprises co-administering one or more additional therapeutic agents to the subject.

In one embodiment, the additional therapeutic agents are one or more of an immunomodulatory agent, anti-inflammatory agents, glucocorticoid, steroid, non-steroidal anti-inflammatory drug, leukotreine antagonist, β2-agonist, anticholinergic agent, sulphasalazine, penicillamine, dapsone, antihistamines, anti-malarial agents, anti-viral agents, anti-proliferation agents, antibiotics, anti-cancer agents (tamoxifen), cholesterol sequestration agents, cholesterol-efflux compounds, PECAM-1 peptides and synthetic PECAM-1 peptides.

In one embodiment, the artherosclerotic disease is one or more of coronary artery disease stroke, vascular disease, peripheral artery disease, called peripheral artery occlusive disease (PAOD), atherogenesis, stenosis, cancer, cancer metastasis, angiogenesis, cerebrovascular disease, diabetes, diabetic retinopathy, and obesity related angiogenesis.

In a further embodiment, the artherosclerotic disease is one or more of artherosclerotic peripheral artery disease (called peripheral artery occlusive disease (PAOD)), coronary artery disease, stroke, hypertension, diabetes, inflammatory vascular disease, multiple sclerosis, systemic sclerosis (SSc), inflammatory bowel disease, Chagas' disease; cancer, solid tumor, ovarian carcinoma, and preeclampsia.

According to one aspect, provides are methods of selecting a subject for treatment of an artherosclerotic disease, comprising detecting the presence or absence of a variation at one or more of amino acid position 432, nucleotide position 373, amino acid position 563, or nucleotide position 1688 of PECAM-1, and correlating an presence of a variation or heterozygous variation with an indication of increased risk of artherosclerotic disease.

22. The method of claim 21, further comprising correlating the absence of a variation with an indication of decreases risk that a subject will develop artherosclerotic disease.

In one embodiment, the detecting comprises PCR methods, immunological methods, sequencing methods, expression level of PECAM-1 gene, expression level of PECAM-1 protein, and enzyme kinetics of PECAM-1.

In one embodiment, the method further comprises administering a therapeutic amount of an anti-artherosclerotic agent to a subject having the presence of a variation or heterozygous variation.

According to one aspect, provided are methods for determining the therapeutic capacity of a candidate anti-artherosclerotic agent in a subject, comprising determining a PECAM-1 genotype status of a subject or a cell of a subject; determining a pre-treatment artherosclerotic disease status in the subject; administering a therapeutically effective amount of a candidate anti-artherosclerotic agent to the subject; and determining a post-treatment artherosclerotic disease status in the subject.

In one embodiment, a modulation of artherosclerotic disease status indicates that the candidate artherosclerotic agent is efficacious.

In another embodiment, the pre-treatment and post-treatment artherosclerotic disease statuses are determined in a diseased tissue.

In a further embodiment, the diseased tissue is one or more of a heart, brain, blood vessels, cerebrospinal fluid, synovial fluid, serum, stem cells, embryonic tissue, and lung tissue

According to one aspect, provided are methods for determining the therapeutic capacity of a candidate artherosclerotic agent, comprising providing a population of cells with a known PECAM-1 genotype status; contacting the cells with a candidate composition, and determining an effect of the candidate artherosclerotic agent on the subject, wherein a decrease in one or more of blood pressure, cholesterol level, blood glucose level, carbon monoxide levels, nitric oxide level, angina, heart attack, abnormal heart rhythms, heart failure, kidney failure, stroke, obstructed peripheral arteries, plaque rupture, tumor metastasis, tumor growth, lung function, cell aggregation, cell migration, total cholesterol (TC); triglyceride (TG); high density lipoprotein cholesterol (HDL-C); low density lipoprotein cholesterol (LDL-C); apolipoprotein A1 (apoA1); apolipoprotein B (apoB); lipoprotein(a) (Lp(a)), sP-selectin, PECAM-1, sPECAM-1, indicates that the candidate composition may be efficacious.

In one embodiment, the method further comprises correlating the effect of the artherosclerotic agent with the genotype.

In one embodiment, the method further comprising determining the PECAM-1 genotype status of the cells prior to or after providing the cells.

According to one aspect, provided are methods of treating a subject suffering from artherosclerotic disease, comprising determining a PECAM-1 genotype status of a subject or a cell of a subject, and administering a therapeutic amount of an artherosclerotic agent to a subject with a heterozygous or a variant genotype.

In one embodiment, the genotype status is determined by PCR methods, immunological methods, sequencing methods, expression level of PECAM-1 gene, expression level of PECAM-1 protein, or enzyme kinetics of PECAM-1.

In one embodiment, the subject is a mammal or a fish.

In one embodiment, the mammal is a human.

In another embodiment, the human is Asian.

In one embodiment, the artherosclerotic agent comprises (see claim 17 above).

In one embodiment, the method further comprises co-administering one or more additional therapeutic agents to the heterozygous or variant subject.

In one embodiment, the method further comprises determining an effect of the artherosclerotic agent on the subject, wherein a decrease in one or more of blood pressure, cholesterol level, blood glucose level, carbon monoxide levels, nitric oxide level, angina, heart attack, abnormal heart rhythms, heart failure, kidney failure, stroke, obstructed peripheral arteries, plaque rupture, tumor metastasis, tumor growth, lung function, cell aggregation, cell migration, total cholesterol (TC); triglyceride (TG); high density lipoprotein cholesterol (HDL-C); low density lipoprotein cholesterol (DL-C); apolipoprotein A1 (apoA1); apolipoprotein B (apoB); lipoprotein(a) (Lp(a)), sP-selectin, PECAM-1, sPECAM-1, indicates that the candidate composition may be efficacious.

In one embodiment, the artherosclerotic disease is one or more of artherosclerotic peripheral artery disease (called peripheral artery occlusive disease (PAOD)), coronary artery disease, stroke, hypertension, diabetes, inflammatory vascular disease, multiple sclerosis, systemic sclerosis (SSc), inflammatory bowel disease, Chagas' disease; cancer, solid tumor, ovarian carcinoma, preeclampsia, lung disease, and cerebrovascular.

According to one aspect, provided are nucleic acid arrays comprising wildtype and variant alleles of PECAM-1.

According to another aspect, provided are isolated cells over-expressing a protein expressed from one or more of a homozygous wild type 125 allele; a homozygous 563 allele; heterozygous variant of the 125 allele; a homozygous variant of the 125 allele; a heterozygous variant of the 563 allele; a homozygous variant of the 563 allele; heterozygous variant of the 125 allele and a heterozygous variant of the 562 allele; heterozygous variant of the 125 allele and a homozygous variant of the 563 allele; a homozygous variant of the 125 allele and a homozygous variant of the 563 allele; a homozygous variant of the 125 allele and heterozygous variant of the 563 allele; or a homozygous wild type 125 allele; a homozygous 563 allele of PECAM-1.

In one embodiment, the cell comprises one or more of PECAM-1CLeu, PECAM-1CSer or PECAM-1CLeu-PECAM-1CSer.

In another embodiment, the cell comprises one or more of PECAM-1CLeu-PECAM-1Gser, PECAM-1Cleu-PECAM-1Aasn, PECAM-1GVal-PECAM-1Gser, or PECAM-1Gval-PECAM-1Aasn.

In a further embodiment, the cell is a Ren cell.

According to one aspect, provided are transgenic animals over-expressing PECAM-1, wherein the animal comprise one or more of a homozygous wild type 125 allele; a homozygous 563 allele; heterozygous variant of the 125 allele; a homozygous variant of the 125 allele; a heterozygous variant of the 563 allele; a homozygous variant of the 563 allele; heterozygous variant of the 125 allele and a heterozygous variant of the 563 allele; heterozygous variant of the 125 allele and a homozygous variant of the 563 allele; a homozygous variant of the 125 allele and a homozygous variant of the 563 allele; a homozygous variant of the 125 allele and heterozygous variant of the 563 allele; or a homozygous wild type 125 allele and a homozygous 563 allele of PECAM-1.

According to one aspect, provided are transgenic animals over-expressing PECAM-1,

wherein the animal comprise one or more of a combined homozygous wild type 125 allele and 563 allele; a homozygous variant of the 125 allele alone; a homozygous variant of the 563 allele alone; a combined homozygous variant of 125 allele of the 563 allele.

In one embodiment, the animal is a mouse, goat, sheep, horse, rabbit, pig, cow, monkey, fish, or mammalian embryo.

According to one aspect, provided are vectors encoding one or more sequences encoding PECAM-1, wherein the PECAM-1 comprises a 125 variant allele; a 563 variant allele, a 125 wild-type allele; or a 563 wild-type allele.

In one embodiment, the vector comprises vPECAM-1GLeu, vPECAM-1GSer or vPECAM-1GLeu-PECAM-1 Gser.

In another embodiment, the vector comprises vPECAM-1CLeu-Gser, vPECAM-1GVal-Gser, vPECAM-1 CLeu-Aasn, or vPECAM-1 GVal-AAsn.

According to one aspect, provided are kits for the assessment of artherosclerotic disease, comprising oligonucleotide probes that differentiate the wild-type and variant alleles of PECAM-1 and instructions for use, wherein the allele amino acid position 432, nucleotide position 373, amino acid position 563, or nucleotide position 1688 of PECAM-1.

In one embodiment, the oligonucleotide probes are one or more of OLA or Taqman probes.

According to one aspect, provided are kits for the assessment of PECAM-1 status, comprising one or more of oligonucleotide primers that amplify from about nucleotide 450 to about nucleotide 400 or from about nucleotide 1663 to about nucleotide 1715 of PECAM-1 and instructions for use.

According to one aspect, provided are kits for the assessment of artherosclerotic disease risk, comprising a nucleic acid array comprising the wildtype and variant alleles of PECAM-1, one or more of oligonucleotide primers that amplify from about nucleotide 450 to about nucleotide 400 or from about nucleotide 1663 to about nucleotide 1715 of PECAM-1, and instructions for use.

These variances may be useful either during the drug development process or in guiding the optimal use of already approved compounds. DNA sequence variances in candidate genes (e.g., genes that may plausibly affect the action of a drug) are analyzed, leading to the establishment of diagnostic tests useful for improving the development of new pharmaceutical products and/or the more effective use of existing pharmaceutical products.

Also, described herein is the identification of gene sequence variances in PECAM-1 that are predictive of drug action and are useful for determining drug efficacy in an subject.

Other embodiments of the invention are disclosed infra.

### BRIEF DESCRIPTION OF THE DRAWING

FIG. 1 depicts the characterization of PECAM-1 Gene/Protein Expression in Transfected Ren Cells. A: PECAM-1 gene expression in transfected Ren cells was characterized by RT-PCR (a) and quantified by real time quantitative RT-PCR (b) after 4 times repeats. PCR cycles demonstrated in the Bar-graph adversely correlated with the number of cDNA copies of PECAM-1 gene. B: PECAM-1 protein mass was determined Western Immunoblot. Ren#1, Ren (−); Ren#2, Ren (+/WT); Ren#5, Ren (+/PM).

FIG. 2 depicts PECAM-1 distribution in membrane/cytosolic and Triton X-100 soluble/insoluble fractions in transfected Ren Cells. A, B, and C are showing representative Western blot images and bar-graphs of PECAM-1 quantifications. Molecular weight of PECAM-1: 130 KDa, β-actin: 43 KDa. Quantification was performed by densitometry scanning of the films (four repeats) from the Western blot of PECAM-1 and β-actin. PECAM-1 was adjusted to β-actin. A: PECAM-1 detected in total cell lysates. B: PECAM-1 distribution in membrane/cytosolic fractions. a-PECAM-1 in cytosol containing small organelles and particles. b-PECAM-1 in cytosol containing large organelles. c-PECAM-1 in cell membrane. C: PECAM-1 detected in Triton X-100 soluble and insoluble fractions. D: Images of PECAM-1 staining with indirect immunofluorescent confocol microscopy. *P<0.05; **P<0.01.

FIG. 3 depicts levels of sPECAM-1 detected in Ren cell culture medium. Ren cell culture medium was concentrated (4:1) by passing through a Millipore YM-10 column. Protein concentration was measured and 10 μl of the concentrated medium was subjected to ELISA. The bar-graph was plotted by adjusting the level of Ren (+/WT) as 100%. *P<0.05

FIG. 4 depicts the results of a Ren cell aggregation assay. About 2×106 of Ren cells, Ren (−), Ren (+/WT) and Ren (+/PM), were seeded in a T75 flask and cultured for 48 hours for aggregation assays. The monodispersed Ren cells suspension (106 cells/ml in HBSS with 1 mM CaCl₂) were prepared. Next, two concentrations of anti-PECAM-1 antibody (JHS-7) (A), and human rcPECAM-1 (B) were added in. Then, cells were transfected to wells in a 24-well tissue culture tray (1 mL per well) and rotated on a gyratory shaker (at 90 rpm) at 37° C. for 20 minutes. Data are expressed as the percent of total cells present in aggregates. *P<0.05, **P<0.01.

FIG. 5 depicts U-937 trans-migration assays. Monolayers of Ren cells were subjected to U-937 cells trans-migration assay. The transmigration of U-937 cells could be observed as early as 2 hours (not shown). At 12 hours the results were more obvious. TEM was determined as a percentage of the Ren (+/WT) and the assay was repeated 3 times each carried out in quadruplicates. *P<0.05.

### DETAILED DESCRIPTION

Disclosed herein is a target gene and variances having utility in pharmacogenetic association studies and diagnostic tests to improve the use of certain drugs or other therapies including, for example, docetaxel and other anti-artherosclerotic agents that may be described in the 1999 Physicians' Desk Reference (53rd edition), Medical Economics Data, 1998, or the 1995 United States Pharmacopeia XIII National Formulary XVIII, Interpharm Press, 1994, or other sources as described below.

As used herein, the term “polymorphic site” refers to a region in a nucleic acid at which two or more alternative nucleotide sequences are observed in a significant number of nucleic acid samples from a population of subjects. A polymorphic site may be a nucleotide sequence of two or more nucleotides, an inserted nucleotide or nucleotide sequence, a deleted nucleotide or nucleotide sequence, or a microsatellite, for example. A polymorphic site may be two or more nucleotides in length, such as 3, 4, 5, 6, 7, 8, 9, 10, 11, 12, 13, 14, 15 or more, 20 or more, 30 or more, 50 or more, 75 or more, 100 or more, 500 or more, or about 1000 nucleotides in length, where all or some of the nucleotide sequences differ within the region. A polymorphic site is often one nucleotide in length, which is referred to herein as a single nucleotide polymorphism (SNP). PECAM-1 polymorphic sites include, e.g., Leu 125 Val (C 373 G) and Ser 563 Asn (G 1688 A).

Where there are two, three, or four alternative nucleotide sequences at a polymorphic site, each nucleotide sequence is referred to as a “polymorphic variant” or “nucleic acid variant.” Where two polymorphic variants exist, for example, the polymorphic variant represented in a minority of samples from a population is sometimes referred to as a “minor allele” and the polymorphic variant that is more prevalently represented is sometimes referred to as a “major allele.” Many organisms possess a copy of each chromosome (e.g., humans), and those subjects who possess two major alleles or two minor alleles are often referred to as being “homozygous” with respect to the polymorphism, and those subjects who possess one major allele and one minor allele are normally referred to as being “heterozygous” with respect to the polymorphism. Individuals who are homozygous with respect to one allele are sometimes predisposed to a different phenotype as compared to subjects who are heterozygous or homozygous with respect to another allele.

The term “genotype” refers to the alleles present in DNA from a subject or patient, where an allele can be defined by the particular nucleotide(s) present in a nucleic acid sequence at a particular site(s). Often a genotype is the nucleotide(s) present at a single polymorphic site known to vary in the human population.

Furthermore, a genotype or polymorphic variant may be expressed in terms of a “haplotype,” which as used herein refers to two or more polymorphic variants occurring within genomic DNA in a group of subjects within a population. For example, two SNPs may exist within a gene where each SNP position includes a cytosine variation and an adenine variation. Certain subjects in a population may carry one allele (heterozygous) or two alleles (homozygous) having the gene with a cytosine at each SNP position. As the two cytosines corresponding to each SNP in the gene travel together on one or both alleles in these subjects, the subjects can be characterized as having a cytosine/cytosine haplotype with respect to the two SNPs in the gene.

As used herein, the term “phenotype” refers to a trait which can be compared between subjects, such as presence or absence of a condition, a visually observable difference in appearance between subjects, metabolic variations, physiological variations, variations in the function of biological molecules, and the like. An example of a phenotype is occurrence of artherosclerotic disease. For example, a phenotype of a homozygous PECAM-1 for Leu125Val variant and for both the Leu 125Val and Ser563Asn variant are at increased risk of artherosclerotic disease, whereas a phenotype of a homozygous wild type does not have increased risk for artherosclerotic disease.

The terms “variant form of a gene,” “variant allele,” or “variant genotype” refer to a specific form of a gene in a population, the specific form differing from other forms of the same gene in the sequence of at least one, and frequently more than one, variant sites within the sequence of the gene. The sequences at these variant sites that differ between different alleles of the gene are termed “gene sequence variances” or “variances” or “variants.” The term “alternative form” refers to an allele that can be distinguished from other alleles by having distinct variances at least one, and frequently more than one, variant sites within the gene sequence. Other terms known in the art to be equivalent include mutation and polymorphism, although mutation is often used to refer to an allele associated with a deleterious phenotype. In the methods utilizing variance presence or absence, reference to the presence of a variance or variances means particular variances, e.g., particular nucleotides at particular polymorphic sites, rather than just the presence of any variance in the gene.

Variances occur in the human genome at approximately one in every 500-1,000 bases within the human genome when two alleles are compared. When multiple alleles from unrelated subjects are compared the density of variant sites increases as different subjects, when compared to a reference sequence, will often have sequence variances at different sites. At most variant sites there are only two alternative nucleotides involving the substitution of one base for another or the insertion/deletion of one or more nucleotides. Within a gene there may be several variant sites. Variant forms of the gene or alternative alleles can be distinguished by the presence of alternative variances at a single variant site, or a combination of several different variances at different sites (haplotypes).

The term “haplotype” refers to a cis arrangement of two or more polymorphic nucleotides, e.g., variances, on a particular chromosome, e.g., in a particular gene. The haplotype preserves information about the phase of the polymorphic nucleotides, that is, which set of variances were inherited from one parent, and which from the other. A genotyping test does not provide information about phase. For example, a subject heterozygous at nucleotide 25 of a gene (both A and C are present) and also at nucleotide 100 (both G and T are present) could have haplotypes 25A-100G and 25C-100T, or alternatively 25A-100T and 25C-100G. Phase can also be predicted statistically based on calculations of linkage frequencies, and the most likely phase can be assessed by such methods as well.

A polymorphic variant may be detected on either or both strands of a double-stranded nucleic acid. For example, a thymine at a particular position in a sequence can be reported as an adenine from the complementary strand. Also, a polymorphic variant may be located within an intron or exon of a gene or within a portion of a regulatory region such as a promoter, a 5′ untranslated region (UTR), a 3′ UTR, and in DNA (e.g., genomic DNA (gDNA) and complementary DNA (cDNA)), RNA (e.g., mRNA, tRNA, and rRNA), or a polypeptide. Polymorphic variations may or may not result in detectable differences in gene expression, polypeptide structure, or polypeptide function.

The terms “disease” or “condition” are commonly recognized in the art and designate the presence of signs and/or symptoms in a subject or patient that are generally recognized as abnormal. Diseases or conditions may be diagnosed and categorized based on pathological changes. Signs may include any-objective evidence of a disease such as changes that are evident by physical examination of a patient or the results of diagnostic tests which may include, among others, laboratory tests to determine the presence of DNA sequence variances or variant forms of certain genes in a patient. Symptoms are subjective evidence of disease or a patients condition, e.g., the patients perception of an abnormal condition that differs from normal function, sensation, or appearance, which may include, without limitations, physical disabilities, morbidity, pain, and other changes from the normal condition experienced by an subject. Artherosclerotic diseases or conditions include, for example, those categorized in standard textbooks of medicine including, without limitation, textbooks of nutrition, allopathic, homeopathic, and osteopathic medicine. In certain aspects, the artherosclerotic disease or condition is selected from one or more of coronary artery disease stroke, vascular disease, peripheral artery disease, called peripheral artery occlusive disease (PAOD), atherogenesis, stenosis, cancer, cancer metastasis, angiogenesis, cerebrovascular disease, diabetes, and obesity related angiogenesis. A subject may suffer from one or more of the artherosclerotic diseases or conditions at once or consecutively. Such artherosclerotic disease and conditions are described, for example, in standard texts such as Harrison's Principles of Internal Medicine (14th Ed) by Anthony S. Fauci, Eugene Braunwald, Kurt J. Isselbacher, et al. (Editors), McGraw Hill, 1997, or Robbins Pathologic Basis of Disease (6th edition) by Ramzi S. Cotran, Vinay Kumar, Tucker Collins & Stanley L. Robbins, W B Saunders Co., 1998, or the Diagnostic and Statistical Manual of Mental Disorders: DSM-IV (.sub.4th edition), American Psychiatric Press, 1994, or other texts described below.

The phrase “suffering from a disease or condition” means that a subject is either presently subject to the signs and symptoms, or is more likely to develop such signs and symptoms than a normal subject in the population. Thus, for example, a subject suffering from a condition can include a developing fetus, a subject to a treatment or environmental condition which enhances the likelihood of developing the signs or symptoms of a condition, or a subject who is being given or will be given a treatment which increase the likelihood of the subject developing a particular condition. Thus, methods of the present invention which relate to treatments of patients (e.g., methods for selecting a treatment, selecting a patient for a treatment, and methods of treating a disease or condition in a patient) can include primary treatments directed to a presently active disease or condition, secondary treatments which are intended to cause a biological effect relevant to a primary treatment, and prophylactic treatments intended to delay, reduce, or prevent the development of a disease or condition, as well as treatments intended to cause the development of a condition different from that which would have been likely to develop in the absence of the treatment.

In certain embodiments, the artherosclerotic disease or condition is one which is treatable by anti-artherosclerotic agents, exercise, dietary intake, weight-management, diabetes management, coronary artery disease stroke, vascular disease, peripheral artery disease, called peripheral artery occlusive disease (PAOD), atherogenesis, stenosis, cancer, cancer metastasis, angiogenesis, cerebrovascular disease, diabetes, and obesity related angiogenesis. Disease include, for example, one or more of coronary artery disease stroke, vascular disease, peripheral artery disease, called peripheral artery occlusive disease (PAOD), atherogenesis, stenosis, cancer, cancer metastasis, angiogenesis, cerebrovascular disease, diabetes, and obesity related angiogenesis.

The term “therapy” refers to a process that is intended to produce a beneficial change in the condition of a mammal, e.g., a human, often referred to as a patient. A beneficial change can, for example, include one or more of restoration of function, reduction of symptoms, limitation or retardation of progression of a disease, disorder, or condition or prevention, limitation or retardation of deterioration of a patient's condition, disease or disorder. Such therapy can involve, for example, nutritional modifications, administration of radiation, administration of a drug, behavioral modifications, and combinations of these, among others.

The terms “drug” and “therapeutic agent,” as used herein refer to a chemical entity or biological product, or combination of chemical entities or biological products, administered to a subject to treat or prevent or control a disease or condition, e.g., an anti-artherosclerotic agent. The chemical entity or biological product is preferably, but not necessarily a low molecular weight compound, but may also be a larger compound, for example, an oligomer of nucleic acids, amino acids, or carbohydrates including without limitation proteins, oligonucleotides, ribozymes, DNAzymes, glycoproteins, lipoproteins, and modifications and combinations thereof. A biological product is preferably a monoclonal or polyclonal antibody or fragment thereof such as a variable chain fragment or single chain antibody, nanobody; cells; or an agent or product arising from recombinant technology, such as, without limitation, a recombinant protein, recombinant vaccine, or DNA construct developed for therapeutic, e.g., human therapeutic, use. The term “drug” may include, without limitation, compounds that are approved for sale as pharmaceutical products by government regulatory agencies (e.g., U.S. Food and Drug Administration (FDA), European Medicines Evaluation Agency (EMEA), and a world regulatory body governing the International Conference of Harmonization (ICH) rules and guidelines), compounds that do not require approval by government regulatory agencies, food additives or supplements including compounds commonly characterized as vitamins, natural products, and completely or incompletely characterized mixtures of chemical entities including natural compounds or purified or partially purified natural products. The term “drug” as used herein is synonymous with the terms “medicine,” “pharmaceutical product,” or “product.” Most preferably the drug is approved by a government agency for treatment of a specific disease or condition. Included are “candidate compounds” or “candidate anti-artherosclerotic agents,” refers to a drug, agent or compound that is under investigation, either in laboratory or human clinical testing for a specific disease, disorder, or condition.

The term “probe,” as used herein, refers to a molecule which detectably distinguishes between target molecules differing in structure. Detection can be accomplished in a variety of different ways depending on the type of probe used and the type of target molecule. Thus, for example, detection may be based on discrimination of activity levels of the target molecule, but preferably is based on detection of specific binding. Examples of such specific binding include antibody binding and nucleic acid probe hybridization. Thus, for example, probes can include enzyme substrates, antibodies and antibody fragments, and nucleic acid hybridization probes. Thus, in preferred embodiments, the detection of the presence or absence of the at least one variance involves contacting a nucleic acid sequence which includes a variant site with a probe, preferably a nucleic acid probe, where the probe preferentially hybridizes with a form of the nucleic acid sequence containing a complementary base at the variance site as compared to hybridization to a form of the nucleic acid sequence having a non-complementary base at the variant site, where the hybridization is carried out under selective hybridization conditions. Such a nucleic acid hybridization probe may span two or more variant sites. Unless otherwise specified, a nucleic acid probe can include one or more nucleic acid analogs, labels or other substituents or moieties so long as the base-pairing function is retained. For example, techniques such as OLA, TAQMAN, and methods described in US Patent Application Publication No. 2004/0121371, which is hereby incorporated by reference, are also useful detection methods according to the methods disclosed herein.

“Genotype status,” as used herein refers to the particular genotype of a subject, a tissue of a subject and/or of a cell of a subject. The genotype may be of just one gene, or may be of many genes. For example, the genotype status may be of PECAM-1 and determined by detecting the presence or absence of a variation at nucleotide position 373 or amino acid position 125 or nucleotide position 1688 or amino acid position 563.

A wildtype genotype status of PECAM-1 is Leu at amino acid 125 and C at nucleotide 373, and a variant PECAM-1 is amino acid Val at 125 and nucleotide G at 373. A wildtype genotype status of PECAM-1 is Ser at amino acid 563 and G at nucleotide 1688, and a variant PECAM-1 is amino acid Asn at 563 and nucleotide A at 1688. A heterozygous status of PECAM-1 is one allele with Leu at amino acid 125 and/or C at nucleotide 373, and on the other allele one variant PECAM-1 with amino acid Val at 125 and/or nucleotide G at 373 (e.g., CG). A heterozygous status of PECAM-1 is one allele with Ser at amino acid 563 and/or G at nucleotide 1688, and on the other allele one variant PECAM-1 with amino acid Asn at 563 and/or nucleotide A at 1688, (e.g., GA). PECAM-1 may be homozygous or heterozygous at both the 125 and 563 alleles or may be homozygous at one and heterozygous at the other allele.

The genotype status may be determined, for example, by biochemical methods, e.g., array based methods, PCR based methods, and other method now known or later developed in the art.

“Anti-artherosclerotic agent,” as used herein is an agent that will reduce, slow, alleviate symptoms of or cause of artherosclerotic disease. Example include, for example, statins, fibrate (Clofibrate, Gemfibrozil (e.g. Lopid®), Fenofibrate Bezafibrate (e.g. Bezalip®), Ciprofibrate (e.g. Modalim®))), aspirin, warfarin, niacin, beta-blockers (acebutolol, atenolol, betaxolol, bisoprolol, esmolol, metoprolol, nebivolol, butoxamine, nadolol, oxprenolol, propranolol, pindolol, sotalol, timolol), calcium channel-blockers, angiotensin-converting enzyme (ACE) inhibitors (e.g., Sulfhydryl-containing ACE inhibitors, e.g., Captopril (Capoten®); Dicarboxylate-containing ACE inhibitors, e.g., Enalapril (Vasotec®/Renitec®), Ramipril (Altace®/Tritace®/Ramace®), Quinapril (Accupril®), Perindopril (Coversyl®), Lisinopril (Lisodur®/Prinivil®/Zestril®); phosphonate-containing ACE inhibitors, e.g., Fosinopril (Monopril®); naturally occurring, e.g., casokinins and lactokinins)), angiotension II receptor blockers, vasodilators, cardiac glycosides/anti-arrhythmics, diuretics (spironolactone, amiloride, triamterene, water, cranberry juice, caffeine, acetazolamide, dorzolamide, furosemide, bumetanide, ethacrynic acid, hydroclilorothiazide, bendroflumethiazide, mannitol, and glucose), cholesterol-lowering drugs (Ezetimibe (Zetia®, Ezemibe®, Ezetrol®)), statins (e.g., atorvastatin (Lipitor®), fluvastatin (Lescol®), lovastatin (Mevacor®, Altocor®, not marketed in the UK), pravastatin (Pravachol®, Selektine®, Lipostat®), rosuvastatin (Crestor®), simvastatin (Zocor®, Lipex®), cerivastatin (Lipobay®, Baycol®)—marketing discontinued in 2001 by the manufacturer (Bayer) due to serious side-effects, especially when used in combination with fibrates, and combination of ezetimibe and simvastatin (Vytorin®)), folic acid, PPAR inhibitors, cholesterol efflux agents.

“Co-administering,” as used herein refers to the administration with another agent, either at the same time, in the same composition, at alternating times, in separate compositions, or combinations thereof.

“One or more additional therapeutic agents,” refers to the selection of additional therapeutic agents that may be co-administered with the anti-artherosclerotic agent are selected from an immunomodulatory agent, anti-inflammatory agents, glucocorticoid, steroid, non-steroidal anti-inflammatory drug, leukotreine antagonist, β2-agonist, anticholinergic agent, sulphasalazine, penicillamine, dapsone, antihistamines, anti-malarial agents, anti-viral agents, anti-proliferation agents, antibiotics, anti-cancer agents (tamoxifen), cholesterol sequestration agents, cholesterol-efflux compounds, PECAM-1 peptides and synthetic PECAM-1 peptides.

As used herein, the terms “tumor” or “cancer” refer to a condition characterized by anomalous rapid proliferation of abnormal cells in a subject. The abnormal cells often are referred to as “neoplastic cells,” which are transformed cells that can form a solid tumor.

As used herein, “assessing the risk of artherosclerotic disease in a subject,” refers to, for example, the determination of the clinical outcome based on percentages of, for example, survival given their genotype and treatment options.

“Assessing the responsiveness of a subject to treatment with an anti-artherosclerotic agent,” may be done by any clinical or biological method. For example, a reduction in blood pressure, cholesterol level, blood glucose level, carbon monoxide levels, nitric oxide level, angina, heart attack, abnormal heart rhythms, heart failure, kidney failure, stroke, obstructed peripheral arteries, plaque rupture, tumor metastasis, tumor growth, lung function, cell aggregation, cell migration, total cholesterol (TC); triglyceride (TG); high density lipoprotein cholesterol (HDL-C); low density lipoprotein cholesterol (LDL-C); apolipoprotein A1 (apoA1); apolipoprotein B (apoB); lipoprotein(a) (Lp(a)), sP-selectin, PECAM-1, sPECAM-1, indicates that the candidate composition may be efficacious and may be followed by diagnostic methods, observation and/or by in vitro cell based methods.

“Providing,” refers to obtaining, by for example, buying or making the, e.g., polypeptide, drug, polynucleotide, probe, and the like. The material provided may be made by any known or later developed biochemical or other technique. For example, polypeptides may be obtained from cultured cells. The cultured cells, for example, may comprise an expression construct comprising a nucleic acid segment encoding the polypeptide.

Cells and/or subjects may be treated and/or contacted with one or more additional anti-artherosclerotic treatments including, surgery, exercise, dietary changes, or other therapy recommended or proscribed by self or by a health care provider.

Identifying a subject in need of such treatment can be in the judgment of a subject or a health care professional and can be subjective (e.g., opinion) or objective (e.g., measurable by a test or diagnostic method).

As used herein, “treating, preventing or alleviating artherosclerotic disease,” refers to the prophylactic or therapeutic use of the therapeutic agents described herein, e.g., anti-artherosclerotic agents.

“Substantially purified” when used in the context of a polypeptide or polynucleotide, or fragment or variant thereof that are at least 60% free, preferably 75% free and more preferably 90% free from other components with which they are naturally associated. An “isolated polypeptide” or “isolated polynucleotide” is, therefore, a substantially purified polypeptide or polynucleotide, respectively.

The term “subject” includes organisms which are capable of suffering from artherosclerotic disease or who could otherwise benefit from the administration of a compound or composition of the invention, such as human and non-human animals. Preferred human animals include human patients suffering from or prone to suffering from artherosclerotic disease or associated state, as described herein. The term “non-human animals” of the invention includes all vertebrates, e.g., mammals, e.g., rodents, e.g., mice, and non-mammals, such as non-human primates, e.g., sheep, dog, cow, chickens, amphibians, reptiles, fish etc.

A method for “predicting” or “diagnosing” as used herein refers to a clinical or other assessment of the condition of a subject based on observation, testing, or circumstances. Various anatomic, physiological & behavioral risk factors for artherosclerosis include, for example, aging; being male; having diabetes or just upper normal blood glucose and insulin levels (e.g., any glycosolated hemoglobin, HbA1c, above 5.0); dyslipidemia (elevated cholesterol or triglyceride levels); having a high blood concentration of low density lipoprotein (LDL, “bad cholesterol”) particles, elevated lipoprotein little a, and very low density lipoprotein (VLDL) particles; having a low concentration of functioning high density lipoprotein (HDL, “good cholesterol”) particles; very high levels of HDL particles enriched in apolipoprotein C-1, VLDL having low levels of apoA-V, elevated levels of homocystiene, e.g., due to dietary deficiencies and/or mutations of the genes involved in homocystiene metabolism, tobacco smoking; high blood pressure; obesity; having close relatives who had heart disease or a stroke at a relatively young age; being physically less active; chronic sub-clinical scurvy; chronic inflammation; elevated fibrinogen blood concentrations; elevated levels of homocystiene; having trouble managing stress; depression, exposure to small pollutant particles, e.g., from industrial waste and gasoline exhausts, increased levels of plasminogen activator inhibitor, eating disorders, and/or habitual over-eaters leading to obesity/diabetes.

“Determining a level of expression” or “determining a genotype,” may be by any now known or hereafter developed assay or method of determining expression level, for example, immunological techniques, PCR techniques, immunoassay, quantitative immunoassay, Western blot or ELISA, quantitative RT-PCR, and/or Northern blot. The level may be of RNA or protein. sequencing, real-time PCR, PCR, allele-specific PCR, Pyrosequencing, SNP Chip technology, or RFLP. One of skill in the art, having the benefit of this disclosure would know how to determine the genotype of PECAM-1.

A sample or samples may be obtained from a subject, for example, by swabbing, biopsy, lavage, phlebotomy or spinal tap. Samples include tissue samples, blood, sputum, bronchial washings, biopsy aspirate, or ductal lavage.

“Therapeutically effective amount,” as used herein refers to an amount of an agent which is effective, upon single or multiple dose administration to the cell or subject, in prolonging the survivability of the patient with such a disorder beyond that expected in the absence of such treatment.

Compositions described herein may be administered, for example, by one or more of systemically, intratumorally, intravascularally, to a resected tumor bed, orally, by inhalation, directly by injection, and targeted delivery, e.g., by the use of liposomes encapsulated with drugs, e.g., coated with antibodies and/or markers specific for tissues/organs/cells.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (e.g., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. The primer must be sufficiently long to prime the synthesis of extension products in the presence of the inducing agent. The exact lengths of the primers will depend on many factors, including temperature, source of primer and the use of the method.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem. 260:2605-2608 (1985); Rossolini et al., Mol. Cell. Probes, 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “polymerase chain reaction” (PCR) refers to the methods of U.S. Pat. Nos. 4,683,195, 4,683,202, and 4,965,188, all of which are hereby incorporated by reference, directed to methods for increasing the concentration of a segment of a target sequence in a mixture of genomic DNA without cloning or purification. As used herein, the terms “PCR product” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “recombinant DNA molecule” as used herein refers to a DNA molecule, which is comprised of segments of DNA joined together by means of molecular biological techniques.

As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, an oligonucleotide having a nucleotide sequence encoding a gene refers to a DNA sequence comprising the coding region of a gene or in other words the DNA sequence, which encodes a gene product. The coding region may be present in either a cDNA or genomic DNA form. Suitable control elements such as enhancers/promoters, splice junctions, polyadenylation signals, etc., may be placed in close proximity to the coding region of the gene if needed to permit proper initiation of transcription and/or correct processing of the primary RNA transcript. Alternatively, the coding region utilized in the vectors of the present invention may contain endogenous enhancers/promoters, splice junctions, intervening sequences, polyadenylation signals, etc., or a combination of both endogenous and exogenous control elements.

The terms “identical” or percent “identity,” in the context of two or more nucleic acids or polypeptide sequences, refer to two or more sequences or subsequences that are the same or have a specified percentage of amino acid residues or nucleotides that are the same (e.g., 60% identity, optionally 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Optionally, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math., 2:482 (1981), by the homology alignment algorithm of Needleman & Wunsch, J. Mol. Biol., 48:443 (1970), by the search for similarity method of Pearson & Lipman, Proc. Natl. Acad. Sci. U.S.A., 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

As used herein, the term “antibody” refers to any molecule which has specific immunoreactivity activity, whether or not it is coupled with another compound such as a targeting agent, carrier, label, toxin, or drug. Although an antibody usually comprises two light and two heavy chains aggregated in a “Y” configuration with or without covalent linkage between them, the term is also meant to include any reactive fragment or fragments of the usual composition, such as Fab molecules, Fab proteins or single chain polypeptides having binding affinity for an antigen. Fab refers to antigen binding fragments. As used herein, the term “Fab molecules” refers to regions of antibody molecules which include the variable portions of the heavy chain and/or light chain and which exhibit binding activity. “Fab protein” includes aggregates of one heavy and one light chain (commonly known as Fab), as well as tetramers which correspond to the two branch segments of the antibody Y (commonly known as F(ab)₂), whether any of the above are covalently or non-covalently aggregated so long as the aggregation is capable of selectively reacting with a particular antigen or antigen family.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with the proteins disclosed herein. The antibodies can be tested for their desired activity using the in vitro assays described herein, or by analogous methods, after which their in vivo therapeutic and/or prophylactic activities are tested according to known clinical testing methods.

The antibodies of the instant invention are raised against the different alleles of PECAM-1, e.g., the wild-type and/or variant alleles of PECAM-1 at nucleotide positions 373 and 1688 or the amino acid positions of 125 and 563. The antibody can be a polyclonal, monoclonal, recombinant, e.g., a chimeric or humanized, fully human, non-human, e.g., murine, single chain antibody, or fully synthetic. Chimeric, humanized, but most preferably, completely human antibodies are desirable for applications which include repeated administration, e.g., therapeutic treatment of human patients, and some diagnostic applications. In a related embodiment, the antibody can be coupled to a toxin and/or a statin or a PPAR inhibitor

### Methods of Selecting Subjects, and Assessing Risks of Treatments

Thus, in one aspect, the invention provides a method for selecting a patient for treatment by determining whether or not a gene or genes in cells of the patient (in some cases including both normal and disease cells) contain at least one sequence variance which is indicative of the disease or condition or the risk of developing the disease or condition. The methods disclosed herein may be used with other genotyping or marker methods (e.g., tumor or disease markers) if necessary. In one embodiment, the at least one variance includes a plurality of variances which may provide a haplotype or haplotypes. Preferably the joint presence of the plurality of variances is indicative of the risk of developing artherosclerotic disease. The plurality of variances may each be indicative of the risk, or the plurality of variances may be indicative of the risk. The plurality of variances may also be combinations of these relationships. The plurality of variances may include variances from one, two, three or more gene loci.

In another aspect, methods of treating artherosclerotic disease comprise determining the genotype status of PECAM-1, and correlating the genotype to the treatment.

The determining may comprise methods including, for example, array based methods, PCR based methods, immunological methods (antibodies, western blots, RIAs, etc), nucleic acid methods (expression level of PECAM-1 alleles), sequencing methods (direct and indirect sequencing of oligonucleotides or nucleic acids and peptides or proteins or Pyrosequencing), protein methods (e.g., expression level of PECAM-1 and/or sPECAM-1), enzyme kinetics of PECAM-1, PCR methods (real-time PCR, allele-specific PCR, reverse-transcriptase PCR, PCR), SNP Chip technology, RFLP and/or other assays described herein.

The genotype status, refers to, for example, the genotype of one or both alleles of a humans PECAM-1 gene. The genotype status of PECAM-1 may comprise determining the identity of one or more of the nucleotide positions 373 or 1688 of PECAM-1 and/or determining the identity of the amino acid positions 125 or 563. The assay may be informative if only one allele is determined. For example, if only one allele is determined and it is wild-type, the assay is informative because wild-type subject will be correlated with a decreased risk of artherosclerotic disease.

Primers for PECAM-1 expression detection include, for example, primer sets 1-4 and fragments and variants thereof.

| | |
|---|---|
| Pair 1: | Forward (5′-ctatcagcctggccctgtag-3′) |
| | Reverse (5′-tattcacgccactgtgtgct-3′) |
| | Length: 504 nucleotide covering the |
| | SNP C + 373G (Leu125Val) at exon3; |
| | and another |
| Pair 2: | Forward (5′-ctatcagcctggccctgtag-3′) |
| | Reverse (5′-tctgttgaaggctgtgcagt-3′) |
| | Length: 399 nucleotides |
| | SNP of G + 1688a (Ser563Asn) at exon 8 |
| Pair 3: | gctgacccttctgctctgtttgagaggtggtgctgacatc |
| | Length: 150 nucleotides |
| Pair 4: | cccgaactggaatcttccttgggtttgccctctttttctc |
| | Length: 651 nucleotides |

“Correlating,” “correlation,” “correlates,” as used herein refer to the establishment of mutual or reciprocal relationship between genotype status and therapeutic efficacy of certain treatments as described herein. That is, correlating refers to relating the genotype status to risk of artherosclerotic disease and/or treatment options.

As used herein, “homozygous variant PECAM-1 genotype status,” refers to the PECAM-one or more of Val at both amino acid positions 125; G at both nucleotide positions 373; Asn at both amino acid positions 563; A at both nucleotide positions 1688; Val at both amino acid positions 125 and Asn at both amino acid positions 563; or G at both nucleotide positions 373 and A at both nucleotide positions 1688. Wildtype PECAM-1 genotype status is one or more of Leu at amino acid position 125, C at nucleotide position 373, Ser at amino acid position 563, or G at nucleotide position 1688. Variant PECAM-1 genotype status is one or more of Val at amino acid position 432, G at nucleotide position 373, Asn at amino acid position 563, or A at nucleotide position 1688. Methods described herein may further comprise administering a therapeutic amount of an anti-artherosclerotic agent to the subject. For example, statins, fibrate, aspirin, warfarin, angiotensin-converting enzyme (ACE) inhibitors, beta-blockers, niacin, beta-blockers, calcium channel-blockers, angiotensin-converting enzyme (ACE) inhibitors, angiotension II receptor blockers, vasodilators, cardiac glycosides/anti-arrhythmics, diuretics, cholesterol-lowering drugs, statins, folic acid, fibrate (Clofibrate, Gemfibrozil (e.g. Lopid®), Fenofibrate, Bezafibrate (e.g. Bezalip®), Ciprofibrate (e.g. Modalim®))), aspirin, warfarin, angiotensin-converting enzyme (ACE) inhibitors, beta-blockers, niacin, beta-blockers (acebutolol, atenolol, betaxolol, bisoprolol, esmolol, metoprolol, nebivolol, butoxamine, nadolol, oxprenolol, propranolol, pindolol, sotalol, timolol), calcium channel-blockers, angiotensin-converting enzyme (ACE) inhibitors (e.g., Sulfhydryl-containing ACE inhibitors, e.g., Captopril (Capoten®); Dicarboxylate-containing ACE inhibitors, e.g., Enalapril (Vasotec®/Renitec®), Ramipril (Altace®/Tritace®/Ramace®), Quinapril (Accupril®), Perindopril (Coversyl®), Lisinopril (Lisodur®/Prinivil®/Zestril®); phosphonate-containing ACE inhibitors, e.g., Fosinopril (Monopril®); naturally occurring, e.g., casokinins and lactokinins)), angiotension II receptor blockers, vasodilators, cardiac glycosides/anti-arrhythmics, diuretics (spironolactone, amiloride, triamterene, water, cranberry juice, caffeine, acetazolamide, dorzolamide, furosemide, bumetanide, ethacrynic acid, hydrochlorothiazide, bendroflumethiazide, mannitol, and glucose), cholesterol-lowering drugs (Ezetimibe (Zetia®, Ezemibe®, Ezetrol®)), statins (e.g., atorvastatin (Lipitor®), fluvastatin (Lescol®), lovastatin (Mevacor®, Altocor®, not marketed in the UK), pravastatin (Pravachol®, Selektine®, Lipostat®), rosuvastatin (Crestor®), simvastatin (Zocor®, Lipex®), cerivastatin (Lipobay®, Baycol®)—marketing discontinued in 2001 by the manufacturer (Bayer) due to serious side-effects, especially when used in combination with fibrates, and combination of ezetimibe and simvastatin (Vytorin®)), folic acid, placitaxel, tamoxifen. The treatment may be individualized to the subject an other anti-artherosclerotic agents may be co-administered.

Methods of assessing the risk of cancer in a subject are also presented and comprise determining the genotype status of PECAM-1, and correlating the genotype status to cancer risk. A subject has increased risk if they are determined to be homozygous for both PECAM-1 variants or for either variant or heterozygous for 373 and homozygous for the variant of 1688 alleles. Subjects are also at risk if they are heterozygous for only 373, homozygous for 373, heterozygous for 1688 or homozygous for 1688.

In some cases, the selection of a method of treatment or assessment, e.g., a therapeutic regimen, may incorporate selection of one or more from a plurality of medical therapies. Thus, the selection may be the selection of a method or methods which is/are more effective or less effective than certain other therapeutic regimens (with either having varying safety parameters). Likewise or in combination with the preceding selection, the selection may be the selection of a method or methods, which is safer than certain other methods of treatment in the patient.

The selection may involve either positive selection or negative selection or both, meaning that the selection can involve a choice that a particular method would be an appropriate method to use and/or a choice that a particular method would be an inappropriate method to use. Stating that the treatment will be effective means that the probability of beneficial therapeutic effect is greater than in a subject not having the appropriate presence or absence of particular variances. A treatment may be contra-indicated if the treatment results, or is more likely to result, in undesirable side effects, an excessive level of undesirable side effects, and/or no beneficial results. A determination of what constitutes excessive side-effects will vary, for example, depending on the disease or condition being treated, the availability of alternatives, the expected or experienced efficacy of the treatment, and the tolerance of the patient. As for an effective treatment, this means that it is more likely that desired effect will result from the treatment administration in a patient with a particular variance or variances than in a patient who has a different variance or variances.

Also in some embodiments, the method of selecting a treatment involves selecting a method of administration of a compound, combination of compounds, or pharmaceutical composition, for example, selecting a suitable dosage level and/or frequency of administration, and/or mode of administration of a compound. The method of administration can be selected to provide better, preferably maximum therapeutic benefit. In this context, “maximum” refers to an approximate local maximum based on the parameters being considered, not an absolute maximum.

Also in this context, a “suitable dosage level” refers to a dosage level which provides a therapeutically reasonable balance between pharmacological effectiveness and deleterious effects. Often this dosage level is related to the peak or average serum levels resulting from administration of a drug at the particular dosage level.

A particular gene or genes can be relevant to the treatment of more than one disease or conditions for example, the gene or genes can have a role in the initiation, development, course, treatment, treatment outcomes, or health-related quality of life outcomes of a number of different diseases, disorders, or conditions. Thus, in preferred embodiments, the disease or condition or treatment of the disease or condition is any which involves arteriosclerosis. As is generally understood, administration of a particular treatment, e.g., administration of a therapeutic compound or combination of compounds, is chosen depending on the disease or condition which is to be treated. Thus, in certain preferred embodiments, the disease or condition is one for which administration of a treatment is expected to provide a therapeutic benefit.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such a treatment may be ineffective in a subgroup that can be identified by the presence of one or more sequence variances or alleles.) “Less effective” means that the treatment results in a therapeutically significant lower level of pharmacological effectiveness and/or a therapeutically greater level of adverse physiological effects, e.g., greater liver toxicity.

Effectiveness is measured in a particular population. In conventional drug development the population is generally every subject who meets the enrollment criteria (e.g., has the particular form of the disease or condition being treated).

As indicated above, in aspects of this invention involving selection of a patient for a treatment, selection of a method or mode of administration of a treatment, and selection of a patient for a treatment or a method of treatment, the selection may be positive selection or negative selection. Thus, the methods can include eliminating a treatment for a patient, eliminating a method or mode of administration of a treatment to a patient, or elimination of a patient for a treatment or method of treatment.

The term “differential” or “differentially” generally refers to a statistically significant different level in the specified property or effect. Thus, subjects with heterozygous or homozygous variants at one or more of 125 and 563 of PECAM-1 are differentially susceptible to artherosclerotic disease. Preferably, the difference is also functionally significant. Thus, “differential binding or hybridization” is a sufficient difference in binding or hybridization to allow discrimination using an appropriate detection technique. Likewise, “differential effect” or “differentially active” in connection with a therapeutic treatment or drug refers to a difference in the level of the effect or activity which is distinguishable using relevant parameters and techniques for measuring the effect or activity being considered. Preferably the difference in effect or activity is also sufficient to be clinically significant, such that a corresponding difference in the course of treatment or treatment outcome would be expected, at least on a statistical basis.

In certain embodiments, a modulation of subject symptoms indicates that the artherosclerotic agent is efficacious. In other embodiments, the pretreatment and post-treatment subject status are determined in a diseased tissue, e.g., white blood cell, blood, lungs, heart, cerebrospinal fluid, saliva, sweat and/or tears.

In other aspects, methods for determining the therapeutic capacity of a candidate artherosclerotic agent for treating artherosclerotic disease are provided and comprise providing a population of cells with a known PECAM-1 genotype; contacting the cells with a candidate composition, and determining effect of the candidate composition on cell migration assays and cell aggregation assays, described infra, wherein a decrease in cell aggregation, migration, angiogenesis, tissue factor release, thrombus formation, and/or apoptosis, indicates that the candidate composition may be efficacious. The method may further comprise correlating the effect with the genotype. It is possible that the efficacy of certain compounds tested will have a correlation to the PECAM-1 genotype.

The screening methods comprise providing a population of cells with known genotype, and the methods may further comprise obtaining a cell or other biological sample from a subject. The cells may also be primary or established cell lines. The method may further comprise determining the genotype of the cells by the methods described herein. Cells may include, for example, white blood cells, vascular cells, muscle cells, neurons, Ren cells, Ren cells, and/or platelets, expressing one or more variants of PECAM-1, PECAM-1 phosphorylation status, (e.g., phosphorylated/dephosphorylated forms of PECAM-1).

### Methods

Single nucleotide polymorphism (SNP) analysis may be done, for example, by parallel screening of SNPs on micro-arrays. Differential hybridization with allele-specific oligonucleotide (ASO) probes is most commonly used in the microarray format (Pastinen et al., Genome Research 2000). The requirement for sensitivity (e.g., low detection limits) has been greatly alleviated by the development of the polymerase chain reaction (PCR) and other amplification technologies which allow researchers to amplify exponentially a specific nucleic acid sequence before analysis (for a review, see Abramson et al., Current Opinion in Biotechnology, 4:41-47 (1993)). Multiplex PCR amplification of SNP loci with subsequent hybridization to oligonucleotide arrays has been shown to be an accurate and reliable method of simultaneously genotyping at least hundreds of SNPs; see Wang et al., Science, 280:1077 (1998); see also Schafer et al., Nature Biotechnology 16:33-39 (1998).

New experimental techniques for mismatch detection with standard probes, as defined in greater detail below, include, for example, OLA, RCA, Invader™, single base extension (SBE) methods, allelic PCR, and competitive probe analysis. In SBE assays, a polynucleotide probe is attached to a support and hybridized to target DNA. See also US Patent Application Publication No. 2004/0121371.

Generally, for SBE assays, probe sets are designed such that the nucleotide at the 3′ end of the probe is either matched or mismatched with the queried base in the target. If the base matches and hybridizes, the DNA polymerase will extend the probe by one base in the presence of four labeled-terminator nucleotides. Alternately, if the 3′ base is mismatched, the DNA polymerase does not extend the probe. Thus, the identity of the SNP or queried base in the target is determined by the probe set that is extended by the DNA polymerase.

Some probes form internal stem-loop structures resulting in target-independent self-extension of the probe thus giving a false positive signal that interferes with determination of the SNP base. The present invention aims to overcome such problems.

The polymerase chain reaction (PCR) is a widely known method for amplifying nucleic acids. Of the PCR techniques, RT-PCR (Reverse Transcription-PCR), competitive RT-PCR and the like are used for detecting and quantifying a trace amount of mRNA, and show their effectiveness.

In recent years, a real-time quantitative detection technique using PCR has been established (TaqMan PCR, Genome Res., 6 (10), 986 (1996), ABI PRISM™. Sequence Detection System, Applied Biosystems). This technique measures the amount of nucleic acids using a particular fluorescent-labeled probe (TaqMan probe). More specifically, this technique utilizes the following principles: For example, a fluorescent-labeled probe having a reporter dye at the 5′ end and a quencher dye at the 3′ end is annealed to the target DNA, and the DNA is subjected to normal PCR As the extension reaction proceeds, the probe is hydrolyzed from the 5′ end by the 5′-3′ exonuclease activity possessed by DNA polymerase. As a result, the reporter dye at the 5′ end is separated from the quencher dye at the 3′ end, thereby eliminating the FRET (Fluorescence Resonance Energy Transfer, the reduction in fluorescence intensity owing to the decrease in the energy level of the reporter dye caused by the resonance of the two fluorescent dyes) effect produced by the spatial proximity between the two dyes, and increasing the fluorescence intensity of the reporter dye that has been controlled by the quencher dye. The target nucleic acid can be selectively quantified and detected in real-time by measuring the increase of the fluorescence intensity.

This technique is advantageous in that it can test various samples simultaneously in a short time, since, unlike the detection and quantification technique using conventional PCR it does not involve complicated steps, such as agarose gel electrophoresis of the amplified product after PCR and analysis of the electrophoresis pattern.

Determining the presence of a particular variance or plurality of variances in a particular gene in a patient can be performed in a variety of ways. In preferred embodiments, the detection of the presence or absence of at least one variance involves amplifying a segment of nucleic acid including at least one of the at least one variances. Preferably a segment of nucleic acid to be amplified is 500 nucleotides or less in length, more preferably 200 nucleotides or less, and most preferably 45 nucleotides or less. Also, preferably the amplified segment or segments includes a plurality of variances, or a plurality of segments of a gene or of a plurality of genes.

Haplotyping test of PECAM-1, for example, requires allele specific amplification of a large DNA segment of no greater than 25,000 nucleotides, preferably no greater than 10,000 nucleotides and most preferably no greater than 5,000, 4000, 3000, 1500, 1000, 500, 250, or 100 nucleotides. Alternatively one allele may be enriched by methods other than amplification prior to determining genotypes at specific variant positions on the enriched allele as a way of determining haplotypes. Preferably the determination of the presence or absence of a haplotype involves determining the sequence of the variant site or sites by methods such as chain terminating DNA sequencing or minisequencing, or by oligonucleotide hybridization or by mass spectrometry.

In another aspect, the invention provides a method for determining a genotype of an subject in relation to one or more variances in one or more of the genes identified in above aspects by using mass spectrometric determination of a nucleic acid sequence which is a portion of a gene identified for other aspects of this invention or a complementary sequence. Such mass spectrometric methods are known to those skilled in the art. In preferred embodiments, the method involves determining the presence or absence of a variance in a gene; determining the nucleotide sequence of the nucleic acid sequence; the nucleotide sequence is 100 nucleotides or less in length, preferably 50 or less, more preferably 30 or less, and still more preferably 20 nucleotides or less. In general, such a nucleotide sequence includes at least one variance site, preferably a variance site which is informative with respect to the expected response of a patient to a treatment as described for above aspects.

In preferred embodiments, the detection of the presence or absence of the at least one variance involves contacting a nucleic acid sequence corresponding to one of the genes identified above or a product of such a gene with a probe. The probe is able to distinguish a particular form of the gene or gene product or the presence or a particular variance or variances, e.g., by differential binding or hybridization. Thus, exemplary probes include nucleic acid hybridization probes, peptide nucleic acid probes, nucleotide-containing probes which also contain at least one nucleotide analog, and antibodies, e.g., monoclonal antibodies, and other probes as discussed herein. Those skilled in the art are familiar with the preparation of probes with particular specificities. Those skilled in the art will recognize that a variety of variables can be adjusted to optimize the discrimination between two variant forms of a gene, including changes in salt concentration, temperature, pH and addition of various compounds that affect the differential affinity of GC vs. AT base pairs, such as tetramethyl ammonium chloride. (See Current Protocols in Molecular Biology by F. M. Ausubel, R. Brent, R. E. Kingston, D. D. Moore, J. D. Seidman, K. Struhl, and V. B. Chanda (editors, John Wiley & Sons.)

In other preferred embodiments, determining the presence or absence of the at least one variance involves sequencing at least one nucleic acid sample. The sequencing involves sequencing of a portion or portions of a gene and/or portions of a plurality of genes which includes at least one variance site, and may include a plurality of such sites. Preferably, the portion is 500 nucleotides or less in length, more preferably 200 nucleotides or less, and most preferably 45 nucleotides or less in length. Such sequencing can be carried out by various methods recognized by those skilled in the art, including use of dideoxy termination methods (e.g., using dye-labeled dideoxy nucleotides) and the use of mass spectrometric methods. In addition, mass spectrometric methods may be used to determine the nucleotide present at a variance site. In preferred embodiments in which a plurality of variances is determined, the plurality of variances can constitute a haplotype or collection of haplotypes. Preferably the methods for determining genotypes or haplotypes are designed to be sensitive to all the common genotypes or haplotypes present in the population being studied (for example, a clinical trial population).

The process of genotyping involves using diagnostic tests for specific variances that have already been identified. It will be apparent that such diagnostic tests can only be performed after variances and variant forms of the gene have been identified. Identification of new variances can be accomplished by a variety of methods, alone or in combination, including, for example, DNA sequencing, SSCP, heteroduplex analysis, denaturing gradient gel electrophoresis (DGGE), heteroduplex cleavage (either enzymatic as with T4 Endonuclease 7, or chemical as with osinium tetroxide and hydroxylamine), computational methods (described in “VARIANCE SCANNING METHOD FOR IDENTIFYING GENE SEQUENCE VARIANCES” filed Oct. 14, 1999, Ser. No. 09/419,705, and other methods described herein as well as others known to those skilled in the art. (See, for example: Cotton, R. G. H., Slowly but surely towards better scanning for mutations, Trends in Genetics 13(2): 43-6, 1997 or Current Protocols in Human Genetics by N. C. Dracoli, J. L. Haines, B. R. Korf, D. T. Moir, C. C. Morton, C. E. Seidman, D. R. Smith, and A. Boyle (editors), John Wiley & Sons.)

In the context of this invention, the term “analyzing a sequence” refers to determining at least some sequence information about the sequence, e.g., determining the nucleotides present at a particular site or sites in the sequence, particularly sites that are known to vary in a population, or determining the base sequence of all of a portion of the particular sequence.

Also usefully provided herein are probes which specifically recognize a nucleic acid sequence corresponding to a variance or variances in a gene as identified in aspects above or a product expressed from the gene, and are able to distinguish a variant form of the sequence or gene or gene product from one or more other variant forms of that sequence, gene, or gene product under selective conditions. Such genes, include, for example PECAM-1, GenBank accession nos.: nucleotide (NM_{—}000442, S66450, S79861, X96849, BD136489, BD136422-88, AJ313330, X96848, AH002931, L34657, and M28526), and protein, (AAB28645, AAC48566, AAA60057, NP_{—}000433, NP_{—}001027550, AAH22512, P16284, and P51866), (the PECAM-1 sequence is provided in Serebruany V L, Gurbel P A. Effect of thrombolytic therapy on platelet expression and plasma concentration of PECAM-1 (CD31) in patients with acute myocardial infarction, Arterioscler Thromb Vasc Biol 1999; 19: 153-8, which is hereby incorporated by reference in its entirety), which are hereby incorporated by reference in their entirety. Those skilled in the art recognize and understand the identification or determination of selective conditions for particular probes or types of probes. An exemplary type of probe is a nucleic acid hybridization probe, which will selectively bind under selective binding conditions to a nucleic acid sequence or a gene product corresponding to one of the genes identified for aspects above. Another type of probe is a peptide or protein, e.g., an antibody or antibody fragment which specifically or preferentially binds to a polypeptide expressed from a particular form of a gene as characterized by the presence or absence of at least one variance. Thus, in another aspect, the invention concerns such probes. In the context of this invention, a “probe” is a molecule, commonly a nucleic acid, though also potentially a protein, carbohydrate, polymer, or small molecule, that is capable of binding to one variance or variant form of the gene to a greater extent than to a form of the gene having a different base at one or more variance sites; such that the presence of the variance or variant form of the gene can be determined. Preferably the probe distinguishes at least one variance described herein.

In one embodiment, the probe is a nucleic acid probe 6, 7, 8, 9, 10, 11, 12, 13, 14, or 15, preferably at least 17 nucleotides in length, more preferably at least 20 or 22 or 25, preferably 500 or fewer nucleotides in length, more preferably 200 or 100 or fewer, still more preferably 50 or fewer, and most preferably 30 or fewer. In preferred embodiments, the probe has a length in a range from any one of the above lengths to any other of the above lengths (including endpoints). The probe specifically hybridizes under selective hybridization conditions to a nucleic acid sequence corresponding to a portion of one of the genes identified in connection with above aspects. The nucleic acid sequence includes at least one and preferably two or more variance sites. Also in preferred embodiments, the probe has a detectable label, preferably a fluorescent label. A variety of other detectable labels are known to those skilled in the art. Such a nucleic acid probe can also include one or more nucleic acid analogs.

In one embodiment, the probes are products of PCR from the primer pairs 1-4 or from a product of various combinations of the primer pairs 1-4. Probes may also be, for example, nucleotides 1-400, 300-475, 350-400, 1200-1700, 1500-1700, 1600-1720, 1650-1690 of PECAM-1.

In connection with nucleic acid probe hybridization, the term “specifically hybridizes” indicates that the probe hybridizes to a sufficiently greater degree to the target sequence than to a sequence having a mismatched base at least one variance site to allow distinguishing such hybridization. The term “specifically hybridizes,” thus refers to the probe hybridizing to the target sequence, and not to non-target sequences, at a level which allows ready identification of probe/target sequence hybridization under selective hybridization conditions. Thus, “selective hybridization conditions” refer to conditions which allow such differential binding. Similarly, the terms “specifically binds” and “selective binding conditions” refer to such differential binding of any type of probe, e.g., antibody probes, and to the conditions which allow such differential binding. Typically hybridization reactions to determine the status of variant sites in patient samples are carried out with two different probes, one specific for each of the (usually two) possible variant nucleotides. The complementary information derived from the two separate hybridization reactions is useful in corroborating the results. Likewise, provided herein are isolated, purified or enriched nucleic acid sequences of 15 to 500 nucleotides in length, preferably 15 to 100 nucleotides in length, more preferably 15 to 50 nucleotides in length, and most preferably 15 to 30 nucleotides in length, which has a sequence which corresponds to a portion of one of the genes identified for aspects above. Preferably the lower limit for the preceding ranges is 17, 20, 22, or 25 nucleotides in length. In other embodiments, the nucleic acid sequence is 30 to 300 nucleotides in length, or 45 to 200 nucleotides in length, or 45 to 100 nucleotides in length. The nucleic acid sequence includes at least one variance site. Such sequences can, for example, be amplification products of a sequence which spans or includes a variance site in a gene identified herein. Likewise, such a sequence can be a primer, or amplification oligonucleotide which is able to bind to or extend through a variance site in such a gene. Yet another example is a nucleic acid hybridization probe comprised of such a sequence. In such probes, primers, and amplification products, the nucleotide sequence can contain a sequence or site corresponding to a variance site or sites, for example, a variance site identified herein. Preferably the presence or absence of a particular variant form in the heterozygous or homozygous state is indicative of the effectiveness of a method of treatment in a patient.

Likewise, the invention provides a set of primers or amplification oligonucleotides (e.g., 2, 3, 4, 6, 8, 10 or even more) adapted for binding to or extending through at least one gene identified herein. For example, primer pairs 1-4.

In reference to nucleic acid sequences which “correspond” to a gene, the term “correspond” refers to a nucleotide sequence relationship, such that the nucleotide sequence has a nucleotide sequence which is the same as the reference gene or an indicated portion thereof, or has a nucleotide sequence which is exactly complementary in normal Watson-Crick base pairing, or is an RNA equivalent of such a sequence, e.g., an mRNA, or is a cDNA derived from an mRNA of the gene.

In the genetic analysis that associated artherosclerotic disease with the polymorphic variants described herein, samples from subjects having artherosclerotic disease and subjects not having artherosclerotic disease were genotyped. The term “genotyped” as used herein refers to a process for determining a genotype of one or more subjects, where a “genotype” is a representation of one or more polymorphic variants in a population. Genotypes may be expressed in terms of a “haplotype,” which as used herein refers to two or more polymorphic variants occurring within genomic DNA in a group of subjects within a population. For example, two SNPs may exist within a gene where each SNP position includes a cytosine variation and an adenine variation. Certain subjects in a population may carry one allele (heterozygous) or two alleles (homozygous) having the gene with a cytosine at each SNP position.

Genotype methods may be used with or other assays, e.g., diagnostic assays. For example, protein levels, (e.g., sP-selectin, PECAM-1, sPECAM-1), lipid levels, (e.g., total cholesterol (TC); triglyceride (TG); high density lipoprotein cholesterol (HDL-C); low density lipoprotein cholesterol (LDL-C); apolipoprotein A1 (apoA1); apolipoprotein B (apoB); lipoprotein(a) (Lp(a)), apolipoprotein C-1, apolipoprotein A-V, cell aggregation assays, cell migration assays, cell adhesion assays, angiogenesis assays, and/or apoptosis assays.

The present invention provides for both prophylactic and therapeutic methods of treating a subject having, or at risk of having artherosclerotic disease or other conditions that are treatable with anti-artherosclerotic agents.

“Pharmaceutically acceptable excipients or vehicles” include, for example, water, saline, glycerol, ethanol, etc. Additionally, auxiliary substances, such as wetting or emulsifying agents, pH buffering substances, liposomes with, e.g., various combinations of phospahtidylcholine, phasphatidylserine, sphingomyelin, cholesterol and the like, may be present in such vehicles.

The therapeutic methods of the invention generally comprise administration of a therapeutically effective amount of a anti-artherosclerotic agent, e.g., a modulator, e.g., an inhibitor or activator, to a subject in need of such treatment, such as a mammal, and particularly a primate such as a human. Treatment methods of the invention also comprise administration of an effective amount of docetaxel to a subject, particularly a mammal such as a human in need of such treatment for an indication disclosed herein.

A variety of anti-artherosclerotic agents can be employed in the present treatment methods. Simple testing, e.g., in a standard artherosclerotic assay can readily identify suitable anti-artherosclerotic agents. Suitable compounds above and other anti-artherosclerotic agents can be readily prepared by known procedures or can be obtained from commercial sources. See, for example, Abe, A. et al., (1992) J. Biochem. 111:191-196; Inokuchi, J. et al. (1987) J. Lipid Res. 28:565-571; Shukla, A. et al. (1991) J. Lipid Res. 32:73; Vunnam, R. R. et al., (1980) Chem. and Physics of Lipids 26:265; Carson, K. et al., (1994) Tetrahedron Lets. 35:2659; and Akira, A. et al., (1995) J. Lipid Research 36:611. Therapies also include those described in U.S. Pat. No. 5,955,443, which is incorporated by reference herein in its entirety.

In the therapeutic methods of the invention, a treatment compound can be administered to a subject in any of several ways. For example, an anti-artherosclerotic agent can be administered as a prophylactic to prevent the onset of or reduce the severity of a targeted condition. Alternatively, a anti-artherosclerotic agent can be administered during the course of a targeted condition.

In other therapeutic methods of the invention, provided are methods of treating a subject suffering from artherosclerotic disease, comprising determining a PECAM-1 genotype status of a subject or a cell of a subject, and administering an anti-artherosclerotic agent to the subject. The genotype status may be determined as described herein.

A treatment compound can be administered to a subject, either alone or in combination with one or more therapeutic agents, as a pharmaceutical composition in mixture with conventional excipient, e.g., pharmaceutically acceptable organic or inorganic carrier substances suitable for parenteral, enteral or intranasal application which do not deleteriously react with the active compounds and are not deleterious to the recipient thereof. Suitable pharmaceutically acceptable carriers include for example, water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances, liposomes and the like which do not deleteriously react with the active compounds.

Such compositions may be prepared for use in parenteral administration, particularly in the form of liquid solutions or suspensions; for oral administration, particularly in the form of tablets or capsules; intranasally, particularly in the form of powders, nasal drops, or aerosols; vaginally; topically e.g. in the form of a cream; rectally e.g. as a suppository; etc. The anti-artherosclerotic agents or activators may also be administered via stent. Exemplary stents are described in US Patent Application Publication Nos: 20050177246; 20050171599, 20050171597, 20050171598, 20050169969, 20050165474, 20050163821, 20050165352, and 20050171593.

The pharmaceutical agents may be conveniently administered in unit dosage form and may be prepared by any of the methods well known in the pharmaceutical arts, e.g., as described in Remington's Pharmaceutical Sciences (Mack Pub. Co., Easton, Pa., 1980). Formulations for parenteral administration may contain as common excipients such as sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of certain anti-artherosclerotic agents.

Other potentially useful parenteral delivery systems include ethylene-vinyl acetate copolymer particles, osmotic pumps, implantable infusion systems, and liposomes. Formulations for inhalation administration contain as excipients, for example, lactose, or may be aqueous solutions containing, for example, polyoxyethylene-9-lauryl ether, glycocholate and deoxycholate, or oily solutions for administration in the form of nasal drops, or as a gel to be applied intranasally. Formulations for parenteral administration may also include glycocholate for buccal administration, methoxysalicylate for rectal administration, or citric acid for vaginal administration. Other delivery systems will administer the therapeutic agent(s) directly at a surgical site, e.g. after balloon angioplasty a anti-artherosclerotic agent may be administered by use of stents.

An anti-artherosclerotic agent can be employed in the present treatment methods as the sole active pharmaceutical agent or can be used in combination with other active ingredients, e.g., anti-neoplastic or other compounds.

The concentration of one or more treatment compounds in a therapeutic composition will vary depending upon a number of factors, including the dosage of the anti-artherosclerotic agent to be administered, the chemical characteristics (e.g., hydrophobicity) of the composition employed, and the intended mode and route of administration. In general terms, one or more than one of the anti-artherosclerotic agents or activators may be provided in an aqueous physiological buffer solution containing about 0.1 to 10% w/v of a compound for parenteral administration.

It will be appreciated that the actual preferred amounts of active compounds used in a given therapy will vary according to e.g. the specific compound being utilized, the particular composition formulated, the mode of administration and characteristics of the subject, e.g. the species, sex, weight, general health and age of the subject. Optimal administration rates for a given protocol of administration can be readily ascertained by those skilled in the art using conventional dosage determination tests conducted with regard to the foregoing guidelines. Suitable dose ranges may include from about 1 μg/kg to about 100 mg/kg of body weight per day.

Therapeutic compounds of the invention are suitably administered in a protonated and water-soluble form, e.g., as a pharmaceutically acceptable salt, typically an acid addition salt such as an inorganic acid addition salt, e.g., a hydrochloride, sulfate, or phosphate salt, or as an organic acid addition salt such as an acetate, maleate, fumarate, tartrate, or citrate salt. Pharmaceutically acceptable salts of therapeutic compounds of the invention also can include metal salts, particularly alkali metal salts such as a sodium salt or potassium salt; alkaline earth metal salts such as a magnesium or calcium salt; ammonium salts such an ammonium or tetramethyl ammonium salt; or an amino acid addition salts such as a lysine, glycine, or phenylalanine salt.

Preferred anti-artherosclerotic agents exhibit significant activity in cell aggregation and cell migration assays, cell aggregation assays, cell adhesion assays, cell migration assays, angiogenesis assays, and/or apoptosis assays. Examples of these assays may be found in, for example, Cir Res 2005, 97:796-804.

One assays measures the migration of cells through endothelial cells. Preferred anti-artherosclerotic agents exhibit significant activity in a cell migration assays when the cells used in the assays are homozygous wild-type PECAM-1, heterozygous PECAM for either or both 373 and 1688, homozygous variant for either or both 373 and 1688 alleles. Preferably, the anti-artherosclerotic agent inhibits cell migration by at least 15 or 25%, preferably at least 50%, relative to a suitable control assay or control cell or variant cells through wild-type cells. In such an assay, between about 0.1 to 100 μM, preferably between about 1 to 50 μM of a desired anti-artherosclerotic agent is used. Exemplary cell migration assays include monitoring the migration of endothelial cells, for example:

a) seeding one cell type on gelatin coated upper wells of Transwell® plats

b) adding a second cell type in upper wells, wherein second cell type are allowed to transmigrate through first cell type monolayers; and

c) counting second cell type that has migrated to the bottom wells.

One assays measures the aggregation of cells. Preferred anti-artherosclerotic agents exhibit significant activity in a cell aggregation assays when the cells used in the assays are homozygous wild-type PECAM-1, heterozygous PECAM for either or both 373 and 1688, homozygous variant for either or both 373 and 1688 alleles. Preferably, the anti-artherosclerotic agent inhibits cell aggregation by at least 15 or 25%, preferably at least 40%, relative to a suitable control assay or control cell or variant cells through wild-type cells. In such an assay, between about 0.1 to 100 μM, preferably between about 1 to 50 μM of a desired anti-artherosclerotic agent is used. Exemplary cell migration assays include monitoring the aggregation of endothelial cells, for example:

a) shake cells in solution;

b) add glutaraldehyde; and

c) quantify aggregation of the cells

Preferred anti-artherosclerotic agents exhibit significant activity in a cell proliferation assays when the cells used in the assays are homozygous wild-type PECAM-1, heterozygous PECAM for either or both 373 and 1688, homozygous variant for either or both 373 and 1688 alleles. Preferably, the anti-artherosclerotic agent inhibits cell proliferation by at least 15 or 25%, preferably at least 50%, relative to a suitable control assay. In such an assay, between about 0.1 to 100 μM, preferably between about 1 to 50 μM of a desired anti-artherosclerotic agent is used. Exemplary cell proliferation assays include counting viable cells and monitoring activity of specified citric acid cycle enzymes such as lactate dehydrogenase.

One assay measures incorporation of one or more detectably-labeled nucleosides into DNA, e.g., by:

a) culturing suitable cells in medium and adding
1) a candidate anti-artherosclerotic agent and
2) a radiolabeled nucleoside such as ³H-thymidine typically in an amount between about 0.1 to 100 μCi;

b) incubating the cells, e.g., for about 6-24 hours, and typically followed by washing; and

c) measuring incorporation of the radiolabeled nucleoside into DNA over that time relative to a control culture that is prepared and incubated under the same conditions as the assay culture but does not include the potential anti-artherosclerotic agent. The measurement can be achieved by several methods including trichloroacetic acid (TCA) precipitation of labeled DNA on filters followed by scintillation counting. See e.g., Chatteree, S., Biochem. Biophys. Res Comm. (1991) 181:554; Chatterjee, S. et al. (1982) Eur. J. Biochem. 120:435 for disclosure relating to this assay.

In the assays described herein, the endothelial cells may be used in angiogenesis assays; endothelial cells and aortic smooth muscle cells may be used in apoptosis assays; platelets may be used in aggregation assays and thrombus formation; mouse models of PECAM-1 mutants with the polymorphisms and genotype distribution described herein may be used in angiogenesis, apoptosis, early and severe arteriosclerosis, plaque development, maturation and rupture, tissue factor release, thrombus formation myocardial infarction, Ca2+ influx/efflux, bleeding time in vivo assays. An example of an apoptosis assay may be found in Methods in Enzymology, Vol 363, pp 284-299 by Martin, S F and Chatterjee, S. In particular see page 287. An example of an angiogenesis assay may be found in Cir Res 200, 97: 796-804

References herein to a “in vitro cell proliferation assay” or other similar phrase refer to an assay that includes the above steps a) through c). One preferred example of a cell proliferation assay uses tumor cells, particularly those obtained from a human, cow or a rabbit. A suitable protocol involves preparing tumor cells according to standard methods and culturing same in microtitre plates in a suitable medium. A desired anti-artherosclerotic agent is then diluted in the medium, preferably to a final concentration of between about 1 to 100 μg, more preferably between about 1 to 50 μg per ml of medium or less followed by an incubation period of between about 1-5 days, preferably about 1 day or less. Following the incubation, a standard cell proliferation can be conducted, e.g., incorporation of tritiated thymidine or lactate dehydrogenase assay as mentioned above. The assays are preferably conducted in triplicate with a variation of between 5% to 10%. See e.g., Ross, R. J. Cell. Biol. (1971) 50:172; Chatterjee, S. et al. (1982) Eur. J. Biochem. 120:435; Bergmeyer, H. V. In Principles of Enzymatic Analysis. (1978) Verlag Chemie, NY.

Methods for determining the therapeutic capacity of a anti-artherosclerotic agent to reduce, halt, or otherwise modify cell growth in a subject comprise determining pre-treatment levels in a subject; administering a therapeutically effective amount of an anti-artherosclerotic agent to the subject; and determining a post-treatment levels in subject. The levels may be of or a decrease in symptoms, for example, blood pressure, cholesterol level, blood glucose level, carbon monoxide levels, angina, heart attack, abnormal heart rhythms, heart failure, kidney failure, stroke, obstructed peripheral arteries, lipid levels, cell migration, cell aggregation, adhesion, angiogenesis, thrombus formation/tissue factor release, and/or apoptosis.

In one embodiment, a decrease in one or more of blood pressure, cholesterol level, blood glucose level, carbon monoxide levels, angina, heart attack, abnormal heart rhythms, heart failure, kidney failure, stroke, obstructed peripheral arteries, cerebro-vascular disease, plaque rupture, and/or tumor growth/metastasis indicates that the agent is efficacious.

Method for determining the therapeutic capacity of a candidate anti-artherosclerotic agent for treating cancer may also comprise providing a population of cells with a known PECAM-1 genotype; contacting the cells with a candidate composition and determining effect of the candidate composition on cell aggregation and/or migration, wherein a decrease in aggregation or migration indicates that the candidate composition may be efficacious.

A method of assessing the therapeutic capacity or efficacy of the treatment in a subject includes determining the pre-treatment status (e.g., by visual inspection of tissue, measurement of one or more of blood pressure, cholesterol level, blood glucose level, carbon monoxide levels, angina, heart attack, abnormal heart rhythms, heart failure, kidney failure, stroke, obstructed peripheral arteries, cerebro-vascular disease, plaque rupture, and/or tumor growth/metastasis) and then administering a therapeutically effective amount of an anti-artherosclerotic agent to the subject. After an appropriate period of time (e.g., after an initial period of treatment) after the administration of the compound, e.g., 2 hours, 4 hours, 8 hours, 12 hours, or 72 hours, the level of one or more of blood pressure, cholesterol level, blood glucose level, carbon monoxide levels, angina, heart attack, abnormal heart rhythms, heart failure, kidney failure, stroke, obstructed peripheral arteries, cerebro-vascular disease, plaque rupture, and/or tumor growth/metastasis is determined again. The modulation of the one or more of blood pressure, cholesterol level, blood glucose level, carbon monoxide levels, angina, heart attack, abnormal heart rhythms, heart failure, kidney failure, stroke, obstructed peripheral arteries, cerebro-vascular disease, plaque rupture, and/or tumor growth/metastasis indicates efficacy of the treatment. The status of the subject may be determined periodically throughout treatment. For example, the subject status may be checked every few hours, days or weeks to assess the further efficacy of the treatment. A decrease in one or more of blood pressure, cholesterol level, blood glucose level, carbon monoxide levels, angina, heart attack, abnormal heart rhythms, heart failure, kidney failure, stroke, obstructed peripheral arteries, cerebro-vascular disease, plaque rupture, and/or tumor growth/metastasis, for example, indicates that the treatment with an agent is efficacious. The method described may be used to screen or select a subject or a compound that may benefit from treatment with an anti-artherosclerotic agent or may be an effective agent, respectively.

A control experiment is generally tailored for use in a particular assay. For example, most control experiments involve subjecting a test sample (e.g., a population of cells or lysate thereof) to medium, saline, buffer or water instead of a potential anti-artherosclerotic agent in parallel to the cells receiving an amount of test compound. A desired assay is then conducted in accordance with the present methods.

The methods described herein and used to develop the methods here can utilize or utilized a variety of different informative comparisons to identify correlations. For example a plurality of pairwise comparisons of treatment response and the presence or absence of at least one variance can be performed for a plurality of patients. Likewise, the method can involve comparing the response of at least one patient homozygous for at least one variance with at least one patient homozygous for the alternative form of that variance or variances. The method can also involve comparing the response of at least one patient heterozygous for at least one variance with the response of at least one patient homozygous for the at least one variance; Preferably the heterozygous patient response is compared to both alternative homozygous forms, or the response of heterozygous patients is grouped with the response of one class of homozygous patients and said group is compared to the response of the alternative homozygous group.

By “prediction of patient risk” is meant a forecast of the patient's likely health status. This may include a prediction of the patient's risk for developing an artherosclerotic disease, rehabilitation time, recovery time, cure rate, rate of disease progression, predisposition for future disease, or risk of having relapse.

By “therapy for the treatment of a disease” is meant any pharmacological agent or drug with the property of healing, curing, or ameliorating any symptom or disease mechanism associated with drug-induced disease, disorder or dysfunction.

By “pathway” or “gene pathway” is meant the group of biologically relevant genes involved in a pharmacodynamic or pharmacokinetic mechanism of drug, agent, or candidate therapeutic intervention. These mechanisms may further include any physiologic effect the drug or candidate therapeutic intervention renders. Included in this are “biochemical pathways” which is used in its usual sense to refer to a series of related biochemical processes (and the corresponding genes and gene products) involved in carrying out a reaction or series of reactions. Generally in a cell, a pathway performs a significant process in the cell.

By “pharmacological activity” used herein is meant a biochemical or physiological effect of drugs, compounds, agents, or candidate therapeutic interventions upon administration and the mechanism of action of that effect.

### Pharmaceutical Compositions

The small molecule, peptide, nucleic acid, and antibody therapeutics described herein may be formulated into pharmaceutical compositions and be provided in kits. The pharmaceutical formulations may also be coated on medical devices or onto nano-particles for delivery.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT, lecithin, propyl gal late, .alpha.-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, intramuscular, intraperotineal, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an antibody or complex of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary paste or liposome.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-inked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert dilutents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of the present invention may be given orally, parenterally, topically, rectally orally, and/or vaginally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 1.0 to about 100 mg per kg per day. An effective amount is that amount that treats cancer or associated disease.

If desired, the effective daily dose of the active compound may be administered as one dose or as, two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition. Moreover, the pharmaceutical compositions described herein may be administered with one or more other active ingredients that would aid in treating a subject having a HIV infection. In a related embodiment, the pharmaceutical compositions of the invention may be formulated to contain one or more additional active ingredients that would aid in treating a subject having a HIV infection or associated disease or disorder.

The antibodies and complexes, produced as described above, can be provided in kits, with suitable instructions and other necessary reagents, in order to conduct immunoassays as described above. The kit can also contain, depending on the particular immunoassay used, suitable labels and other packaged reagents and materials (e.g., wash buffers and the like). Standard immunoassays, such as those described above, can be conducted using these kits. The pharmaceutical compositions can be included in a container, pack, kit or dispenser together with instructions, e.g., written instructions, for administration, particularly such instructions for use of the antibody or complex to treat or prevent cancer or associated disease. The container, pack, kit or dispenser may also contain, for example, one or more additional active ingredients that would aid in treating a subject having aberrant cell proliferation.

The therapeutic agents described herein, e.g., anti-artherosclerotic agents, are formulated into pharmaceutical preparations for administration.

Additional therapeutic agents may include, but are not limited to, immunomodulatory agents, anti-inflammatory agents (e.g., adrenocorticoids, corticosteroids (e.g., beclomethasone, budesonide, flunisolide, fluticasone, triamcinolone, methlyprednisolone, prednisolone, prednisone, hydrocortisone), glucocorticoids, steroids, non-steriodal anti-inflammatory drugs (e.g. aspirin, ibuprofen, diclofenac, and COX-2 inhibitors), and leukotreine antagonists (e.g. montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents, and antibiotics (e.g., dactinomycin (formerly actinomycin), bleomycin, erythomycin, penicillin, mithramycin, anthramycin (AMC)).

### Antibodies

Antibodies useful in the methods described herein are antibodies specific for and can distinguish alleles of PECAM-1, for example, can distinguish between PECAM-1 wild-type and 125Val and wild-type and 563Asn. Methods of generating antibodies useful in the methods described herein are described more fully below.

Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240: 1041-1043; Liu et al. (1987) Proc. Natl. Acad. Sci. USA 84: 3439-3443; Liu et al. (1987) J. Immunol. 139: 3521-3526; Sun et al. (1987) Proc. Natl. Acad. Sci. USA 84: 214-218; Nishimura et al. (1987) Canc. Res. 47: 999-1005; Wood et al. (1985) Nature 314: 446-449; and Shaw et al. (1988) J. Natl. Cancer Inst. 80: 1553-1559); Morrison, S. L. (1985) Science 229: 1202-1207; Oi et al. (1986) BioTechniques 4: 214; Winter U.S. Pat. No. 5,225,539; Jones et al. (1986) Nature 321: 552-525; Verhoeyan et al. (1988) Science 239: 1534; and Beidler et al. (1988) J. Immunol. 141: 4053-4060.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar (1995) Int. Rev. Immunol. 13: 65-93); and U.S. Pat. Nos. 5,625,126; 5,633,425; 5,569,825; 5,661,016; and 5,545,806. In addition, companies such as Abgenix, Inc. (Fremont, Calif.) and Medarex, Inc. (Princeton, N.J.), can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994) Bio/Technology 12: 899-903).

The term “monoclonal antibody” as used herein refers to an antibody obtained from a substantially homogeneous population of antibodies, e.g., the subject antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567; and Morrison et al., Proc. Natl. Acad. Sci. USA 81: 6851-6855 (1984)).

The present monoclonal antibodies can be made using any procedure which produces monoclonal antibodies. For example, monoclonal antibodies of the invention can be prepared using hybridoma methods, such as those described by Kohler and Milstein, Nature, 256: 495 (1975). In a hybridoma method, a mouse or other appropriate host animal is typically immunized with an immunizing agent to elicit lymphocytes that produce antibodies that will specifically bind to the immunizing agent.

The monoclonal antibodies also can be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of antibodies). Libraries of antibodies or active antibody fragments also can be generated and screened using phage display techniques, e.g., as described in U.S. Pat. No. 5,804,440 to Burton et al. and U.S. Pat. No. 6,096,551 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in International Patent Application Publication No. WO 94/29348, published Dec. 22, 1994, and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-lining antigen.

As used herein, the term “antibody or fragments thereof” encompasses chimeric antibodies and hybrid antibodies, with dual or multiple antigen or epitope specificities, single chain antibodies and fragments, such as F(ab′)2, Fab′, Fab, scFv and the like, including hybrid fragments. Thus, fragments of the antibodies that retain the ability to bind their specific antigens are provided. For example, fragments of antibodies which maintain HIV gp120 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York (1988)). Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase bio-longevity, to alter secretory characteristics; etc. In any case, the antibody or antibody fragment must possess a bioactive property, such as specific binding to its cognate antigen. Functional or active regions of the antibody or antibody fragment can be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment (Zoller, M. J. Curr. Opin. Biotechnol. 3: 348-354 (1992)).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods of the invention serves to lessen the chance that an antibody administered to a human will evoke an undesirable immune response.

Human antibodies also can be prepared using any other technique. Examples of techniques for human monoclonal antibody production include those described by Cole et al. (Monoclonal Antibodies and Cancer Therapy, Alan R. Liss, p. 77 (1985)) and by Boerner et al. (J. Immunol. 147(1): 86-95 (1991)). Human antibodies (and fragments thereof) also can be produced using phage display libraries (Hoogenboom et al., J. Mol. Biol. 227: 381 (1991); Marks et al., J. Mol. Biol. 222: 581 (1991)).

Human antibodies also can be obtained from transgenic animals. For example, transgenic, mutant mice that can produce a full repertoire of human antibodies in response to immunization have been described (see, e.g., Jakobovits et al., Proc. Natl. Acad. Sci. USA 90: 2551-255 (1993); Jakobovits et al., Nature 362: 255-258 (1993); and Bruggermann et al., Year in Immunol. 7: 33 (1993)). Specifically, the homozygous deletion of the antibody heavy chain joining region (J(H) gene in these chimeric and germ-line mutant mice results in complete inhibition of endogenous antibody production, and the successful transfer of the human germ-line antibody gene array into such germ-line mutant mice results in the production of human antibodies upon antigen challenge.

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

Methods for humanizing non-human antibodies are well-known in the art. For example, humanized antibodies can be generated according to the methods of Winter and co-workers (Jones et al., Nature 321: 522-525 (1986); Riechmann et al., Nature 332: 323-327 (1988); and Verhoeyen et al., Science 239: 1534536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### Kits

The oligonucleotide probes may be one or more of OLA, or Taqman.

The kits may comprise oligonucleotide primes that amplify from about nt 300 to about nt 1700 portion of PECAM-1 and instructions for use. The primers may be labeled.

In another aspect, kits for the assessment of cancer treatment options are provided and comprise an array and/or microarray, oligonucleotide primes that amplify from about nucleotide 300 to about nucleotide 400 portion of PECAM-1 and instructions for use. Alternately or in addition, primers may be provided that amplify from about nucleotide 1600 to about nucleotide 1700 of PECAM-1, from about nucleotide 1650 to about nucleotide 1700 of PECAM-1, from about nucleotide 350 to about nucleotide 400 of PECAM-1, from about nucleotide 360 to about nucleotide 390 of PECAM-1, or other portion that one of skill in the art would determine necessary or adequate to amplify and detect the genotype status using array or microarray technology.

In another aspect, a kit for the assessment of cancer treatment options are provided and comprise antibodies that distinguish the wild type and variant PECAM-1 alleles. In one embodiment, the kits comprise one or more of primer pairs 1-4 or combinations of primer pairs 1-4.

Optionally the kits may comprise instructions for use.

The kits described above may further contain enzymes, buffers, labeling agents, and/or pharmaceutical compositions for treatment.

In another aspect, the invention provides a kit containing at least one probe or at least one primer (or other amplification oligonucleotide) or both (e.g., as described above) corresponding to PECAM-1 or other determinants of artherosclerotic disease. The kits are preferably adapted and configured to be suitable for identification of the presence or absence of a particular variance or variances, which can include or consist of a nucleic acid sequence corresponding to a portion of a gene. A plurality of variances may comprise a haplotype of haplotypes. The kit may also contain a plurality of either or both of such probes and/or primers, e.g., 2, 3, 4, 5, 6, or more of such probes and/or primers. Preferably the plurality of probes and/or primers are adapted to provide detection of a plurality of different sequence variances in a gene or plurality of genes, e.g., in 2, 3, 4, 5, or more genes or to amplify and/or sequence a nucleic acid sequence including at least one variance site in a gene or genes.

Preferably one or more of the variance or variances to be detected are correlated with likelihood of developing a disease. In preferred embodiments, the kit contains components (e.g., probes and/or primers) adapted or useful for detection of a plurality of variances (which may be in one or more genes) indicative of the likelihood of developing an artherosclerotic disease. It may also be desirable to provide a kit containing components adapted or useful to allow detection of a plurality of variances indicative of the effectiveness of a treatment or treatment against a plurality of diseases. The kit may also optionally contain other components, preferably other components adapted for identifying the presence of a particular variance or variances. Such additional components can, for example, independently include a buffer or buffers, e.g., amplification buffers and hybridization buffers, which may be in liquid or dry form, a DNA polymerase, e.g., a polymerase suitable for carrying out PCR (e.g., a thermostable DNA polymerase), and deoxy nucleotide triphosphates (dNTPs). Preferably a probe includes a detectable label, e.g., a fluorescent label, enzyme label, light scattering label, or other label. Preferably the kit includes a nucleic acid or polypeptide array on a solid phase substrate. The array may, for example, include a plurality of different antibodies, and/or a plurality of different nucleic acid sequences. Sites in the array can allow capture and/or detection of nucleic acid sequences or gene products corresponding to different variances in one or more different genes. Preferably the array is arranged to provide variance detection for a plurality of variances in one or more genes which correlate with the effectiveness of one or more treatments of one or more diseases, which is preferably a variance as described herein.

The kit may also optionally contain instructions for use, which can include a listing of the variances correlating with a particular treatment or treatments for a disease or diseases and/or a statement or listing of the diseases for which a particular variance or variances correlates with a treatment efficacy and/or safety.

Preferably the kit components are selected to allow detection of a variance described herein, and/or detection of a variance indicative of a treatment, e.g., administration of a drug, pointed out herein.

Additional configurations for kits of this invention will be apparent to those skilled in the art.

The invention also includes the use of such a kit to determine the genotype(s) of one or more subjects with respect to one or more variance sites in one or more genes identified herein. Such use can include providing a result or report indicating the presence and/or absence of one or more variant forms or a gene or genes which are indicative of the effectiveness of a treatment or treatments.

All documents mentioned herein are incorporated by reference herein in their entirety.

### EXAMPLES

The present invention is further illustrated by the following non-limiting examples.

### Example 1

### Research Subjects

Unrelated patients (137 with coronary artery disease (CAD)) who were consecutively referred to National University Hospital of Singapore between 2001 and 2003 were studied. The CAD patients were angiographically defined (having 1, 2, or 3 major epicardial coronary arteries with (≧70% luminal stenosis). Among CAD patients, 5.7% had single vessel disease, 18.9% had double disease and 75.4% had triple vessel disease. None of the CAD patients recruited in the study had acute myocardial infarction. Controls, 110 non-CAD, were recruited from the same period were volunteers by advertisement who did not have a history or clinical evidence of CAD. Further they were confirmed free of CAD by treadmill test.

Most of patients and controls were of South Indian origin in Singapore and have settled in Singapore for over 3 generations. The participants were interviewed in details, and data on smoking habits, hypertension, and diabetes were recorded. Individuals were defined as hypertensive if their blood pressure was >140/90 mmHg or if they were receiving any anti-hypertensive treatment. Individuals with a history of diabetes or those receiving any anti-diabetic medication were considered to be diabetic. Smoker definition included both ex-smokers and active smokers. Both patients and controls with age>70 years, familial hypercholesterolemia, or thyroid, kidney or liver disease or autoimmune disease were excluded from the study.

### Screening of PECAM-1 Gene Polymorphisms

Total blood (15 mL) was obtained from subjects with overnight fasting (12 hours). Genomic DNA was isolated from the white blood cell pellets with a protocol modified from Blin and Stafford²². We have selected two SNPs in the coding sequence, C+373G (Leu125Val) at exon 3 and G+688A (Ser563Asn) at exon 8 reported previously^{17,18,23,24} in polymorphism screening. A polymerase chain reaction (PCR)-restriction fragments length polymorphism (RFLP) procedure was adopted. Based on published sequence of PECAM-1 gene²⁵, PCR primer pairs were designed to generate two DNA fragments covering the above-mentioned SNPs. A pair of oligonucleotide primers, forward (5′-ctatcagcctggccctgtag-3′)/reverse (5′-tattcacgccactgtgtgct-3′) with the product size of 504 nucleotide covering the SNP C+373G (Leu125Val) at exon3; and another pair, forward (5′-ctatcagcctggccctgtag-3′)/reverse (5′-tctgttgaaggctgtgcagt-3′) with the product size of 399 nucleotide covering the SNP of G+1688a (Ser563Asn) at exon 8 were synthesized. The conditions for PCR were: 1) 95° C. for 4 minutes; 2) 95° C. for 30 seconds; 62° C. for 45 seconds and 72° C. for 60 seconds and repeat for 30 cycles; and 3) 72° C. for 7 minutes. PCR product was ethanol precipitated and digested with Pvu II (New England Biolabs, CAG/CTG, from +370 to +375) and Nhe I (New England Biolabs, GCTAG/C, from +1684 to +1689) based on the single nucleotide substitution at C+373G and G+1688A, respectively. Digested PCR products were subjected to agarose gel electrophoresis. Genotyping results from the 15 samples representing 3 genotypes were confirmed by direct sequencing of PCR products using DNA sequencer^{26,27}. Levels of sPECAM-1 and soluble P-selectin (sP-selectin) were measured by enzyme-linked immunosorbent assay (ELISA), according to manufacturer's instruction. ELISA kits were purchased from Bender MedSystem (MedSystems Diagnostics GmbHRennweg 95bA-1030 Vienna, Austria). Lipid panel (lipids, cholesterols and lipoproteins) was determined by routine analytical methods at the pathology department of National University Hospital²⁸.

### Statistical Analysis

χ²-test was used to compare categorical variables, and because of skewed distribution, sP-selectin, the value was expressed as median (25^{th}/75^{th} interquartiles) and compared by Mann-Whitney U test. Other continuous variables were expressed as mean and standard deviation and significance of differences between two groups was assessed by Student's T test. Hardy-Weinberg equilibrium was analyzed by χ2-test for the frequencies of the PECAM-1 genotypes [Weir, B. S. (1996) Genetic Data Analysis II: Methods for Discrete Population Genetics Data. Sinauer, Sunderland, Mass.]. Pearson or Spearman correlation coefficients were computed to assess the association between parameters according to the status of distribution. A p value of less than 0.05 was considered as significant. All computations were performed with Statistical Package for Social Sciences (SPSS,) version 10 (Chicago, Ill.).

### Demographic Details of Research Subjects

As shown in Table I, patients with CAD were older, more likely to be males. The occurrence of diabetes mellitus, smoking, and hypertension were also higher in CAD patients than that in controls. Moreover, patients with CAD had higher levels of triglyceride (TG), higher ratio of total cholesterol (TC) to HDL-C, but lower levels of HDL-C and apoA1, as well as lower ratio of apolipoprotein A1 (apoA1) to apolipoprotein B (apoB). The levels of TC, LDL-C and apoB were lower in CAD compared with controls. There was no difference in lipoprotein (a) (Lp(a)) between two groups (Table II).

### Genotyping for C+373G (Leu125Val) and G+1688A (Ser563Asn) Polymorphism

The presence of two SNPs, C+373G (Leu125Val) at exon 3 and G+1688A (Ser563Asn) at exon 8 were confirmed in our subjects. The genotype frequencies were in agreement with those predicted by the Hardy-Weinberg equilibrium. It was found that a significant association between the genotype distributions of C+373G (Leu125Val) polymorphism and CAD (p=0.009), and G allele frequency was also significantly higher in CAD patients than in controls (p=0.008) (Table IIIA). After adjusting for other risk factors for CAD including age, gender, smoking, hypertension, diabetes, the level of TC and HDL-C by multivariate logistic regression test, GG homozygous was significantly associated with CAD compared with CC plus CG genotypes (a recessive model of inheritance was assumed, odds ratio (95% confidence interval):1.123 (1.060-1.190), p<0.05)). However, the genotype distribution of G+1688A (Ser563Asn) polymorphism did not significantly differ between two groups (p=0.148), and though the frequency of A allele was higher in CAD patients than in control, the difference did not reach significance (p=0.058) (Table III B). The combined effect of two gene polymorphisms was also studied and the results showed that the combination of CG+GG (for Leu125Val) and GA+AA (for Ser563Asn) was significantly increased in patients compared to the controls (67.8% and 51.5% respectively, p=0.014). There was no significant association for Leu125Val or Ser563Asn polymorphisms with the number of affected vessels (both p>0.05).

Plasma sPECAM-1 Level was Elevated in Indian CAD Patients

Patients had significantly higher sPECAM-1 level compared with controls (71.92±25.62 ng/ml vs. 62.77±25.46 ng/ml, p=0.006). The odds ratio (95% confidence interval) was 1.19 (1.07-1.43), p<0.05 after controlling for other risk factors for CAD including age, gender, smoking, hypertension, diabetes, the level of TC and HDL-C by multivariate logistic regression test. The levels of sPECAM-1 did not differ among subjects with different genotypes (p>0.05). Also, there was no significant association between sPECAM-1 levels and the number of affected vessels, p>0.05. Soluble PECAM-1 levels were positively correlated with sP-selectin (r=0.314, p=0.005). Also there were weak associations between sPECAM-1 and TG, LDL-C, HDL-C and apoA1 (r=0.134, r=0.173, r=−0.133, and r=−0.144 respectively, all p<0.05).

Plasma sP-Selectin Level was Elevated in Indian CAD Patients

There was a significant increase in sP-selectin in CAD patients in comparison with controls (median (25^{th}/75^{th} interquartiles): 276.02 (186.19/452.84) ng/ml vs. 166.36 (112.72/228.83) ng/ml respectively, p=0.001). Levels of sP-selectin negatively correlated with HDL-C and apoA1 (r=−0.358, p=0.002, and r=−0.273, p=0.002 respectively).

### PECAM-1 Genotypes and Other Confounders Among CAD Group.

Among CAD group, the genotypes and allele frequencies of Leu125Val were not significantly associated with gender, smoking, diabetes and hypertension. Table IV. Neither did genotypes of Ser563Asn (data not shown). The Leu125Val polymorphism and Ser563Asn located at the 1^{st} and 6th (Ig)-like domains, respectively.

It was found that Leu125Val polymorphism is significantly associated with CAD in Indian patients. It was observed that significant correlation between genotype distribution of Leu125Val polymorphism and CAD (p=0.009) and G allele frequency was significantly higher in CAD patients than in controls (p=0.008).

Only a few studies on PECAM-1 gene polymorphism and CAD have been reported in Caucasians and Japanese. In the German population, Wenzel et al¹⁷ reported that in 103 healthy controls and in 98 patients (Caucasians) with more than 50% stenosis, the allele frequencies of the Leu125Val polymorphism were 0.49/0.51 in controls and 0.35/0.65 in patients (P<0.01) and the allele frequencies of the Ser563Asn polymorphism were 0.50/0.50 in controls and 0.37/0.63 in patients (P<0.05). Without wishing to be bound by any theory, since the interaction/activation of PECAM-1 is mainly via homophilic binding with its 1^{st} extracellular Ig-like domains^{10,11}, the Leu125Val polymorphism might affect the homophilic binding capability, and therefore might influence monocyte/endothelial interaction during the early development of artherosclerotic plaques. On the other hand, the function of the 6^{th} Ig-like domain of PECAM-1 in which Ser563Asn located is less understood, and it could be implicated in calcium homeostasis 19 and monocyte passage through extracellular matrix (interstitial migration) prior to TEM (diapedesis)³⁰

Although an association between the PECAM-1 polymorphisms and CAD follows the same pattern in Indian population as that in other populations, unique allele frequencies of the above two polymorphisms in Indian population as compared to other populations were observed. In the case of Leu125Val, the allele frequencies of C and G are 0.517/0.483 in Chinese controls (Wei, et al, unpublished data) in contrast with 0.664/0.336 in Indian controls, and 0.401/0.599 in Chinese CAD (Wei, et al, unpublished data, not shown) in contrast with 0.536/0.464 in Indian CAD. Other populations such as German¹⁷ and Japanese¹⁸ have the allele distributions similar to Chinese population. The results suggest the frequency of G allele is much lower in Indian population compared with that in other populations. Similarly a striking lower frequency of A allele for Ser563Asn polymorphism was observed in Asian Indian CAD population in general. This funding is interesting, however, in terms of associations of PECAM-1 polymorphisms with CAD. Without wishing to be bound by theory, PECAM-1 interacts with other risk factors such as adult-onset diabetes, low HDL-C, increased Lp (a) levels, high triglycerides, low apoA-V as well as low birth weight^{31 32} accompanied by increase in apoC-1 particles in VLDL or HDL, increased plasminogen activator inhibitor, tissue factor, myeloperoxidase, caspase S levels, etc and collectively contribute to the early onset of CAD in these populations. Soluble PECAM-1 was higher in CAD patients than in controls in Indian population. Similar results were obtained from our study in Chinese CAD patients (Wei et al, unpublished data, not shown). Since almost all patients had at least two vessels affected (more than 70% stenosis), the results suggested that the sPECAM-1 level increased in severe coronary stenosis in Indian CAD patients. The difference in sPECAM-1 between CAD patients and controls is small. Given the roles that PECAM-1 plays in endothelial dysfunction and vascular inflammation, sPECAM-1 level serve as a useful marker to monitor the individualized pathological changes and evaluate the effect of endothelial protective therapy. Moreover, since sPECAM-1 was is positively correlated with sP-selectin, a marker of platelet activation, PECAM-1 might be involved in platelet activation and perhaps related to thrombosis.

In addition, weak correlation was found between sPECAM-1 levels and lipid panel. The levels of sPECAM-1 were positively correlated with TG, LDL-C, while it was negatively correlated with HDL-C and apoA1. Similarly, sP-selectin were also negatively correlated with HDL-C and apoA1. The relationship between lipid and cell adhesion molecules might suggest that that serum lipid level might influence cell adhesion molecules expression. High TG, low HDL-C, as well as high Lp (a) are the typical lipid disorder for Indian CAD patients. As expected, higher levels of TG, higher ratio of TC to HDL-C, but lower levels of HDL-C and apoA1, as well as lower ratio of apoA1 to apoB in Indian CAD patients compared with controls was found. However, a higher Lp (a) level in CAD patients was not found.

The Leu125Val polymorphism of PECAM-1 and the level of sPECAM-1 are correlated with CAD. In addition, a unique pattern of allele frequencies of PECAM-1 polymorphisms was observed in Asian Indian population. PECAM-1 plays an important role in thrombosis and the development of atherosclerosis in Asian Indians.

TABLE I
Demographic details of research subjects

| | Controls | CAD patients |
|---|---|---|
| | (n = 110) | (n = 137) |
| Age (yrs), mean ± | 52.88 ± 10.08 | 60.28 ± 10.42*** |
| SD | | |
| Sex (% of male) | 41.8% | 81.8%*** |
| Diabetes Mellitus | 11.8% | 63.5%*** |
| Smoking | 7.3% | 38.7%* |
| Hypertension | 15.5% | 60.6%*** |

CAD = coronary artery disease;
*P < 0.05,
***P < 0.001

TABLE II
Lipid panel between two groups

| | Control (n = 110) | CAD (n = 137) |
|---|---|---|
| TG (mM) | 1.42 ± 0.72 | 1.73 ± 0.92** |
| TC (mM) | 5.45 ± 1.22 | 4.35 ± 1.12*** |
| HDL-C (mM) | 1.28 ± 0.39 | 0.93 ± 0.28*** |
| LDL-C (mM) | 3.55 ± 0.99 | 3.21 ± 0.89* |
| TC/HDL-C | 4.53 ± 1.39 | 4.98 ± 1.65* |
| ApoA1 (mg/dl) | 145.47 ± 38.86 | 113.50 ± 28.29*** |
| ApoB (mg/dl) | 109.00 ± 36.03 | 97.27 ± 27.48** |
| ApoA1/apoB | 1.59 ± 1.15 | 1.30 ± 0.83* |
| Lp (a) (mg/dl) | 30.67 ± 27.74 | 30.00 ± 27.11 |

CAD = coronary artery disease; TC = total cholesterol; TG = triglyceride; HDL-C = high density lipoprotein cholesterol; LDL-C = low density lipoprotein cholesterol; apoA1 = apolipoprotein A1; apoB = apolipoprotein B; Lp(a) = lipoprotein(a); Values are mean ± SD,
*P < 0.05,
**P < 0.01,
***P < 0.001

TABLE III
Genotypic distributions of the C+373G (Leu125Val) and G+1688A
(Ser563Asn) polymorphism in controls and CAD patients
A: C+373G (Leu125Val)

| C+373G (Leu125Val) | Patients | Control | P |
|---|---|---|---|
| Genotype frequency | N = 137 | N = 110 | |
| CC (%) | 23.4 | 42.7 | |
| CG (%) | 60.6 | 47.3 | |
| GG (%) | 16.0 | 10 | =0.009 |
| Allele frequency | 2n = 274 | 2n = 220 | |
| C allele (%) | 0.536 | 0.664 | |
| G allele (%) | 0.464 | 0.336 | =0.008 |

B: G+1688A (Ser563Asn)

| G+1688A (Ser563Asn) | Patients | Control | P |
|---|---|---|---|
| Genotype frequency | N = 137 | N = 110 | |
| GG (%) | 26.3 | 37.3 | |
| GA (%)_{—} | 53.3 | 49.1 | |
| AA (%) | 20.4 | 13.6 | =0.148 |
| Allele frequency | 2n = 274 | 2n = 220 | |
| G allele (%) | 0.529 | 0.618 | |
| A allele (%) | 0.471 | 0.382 | =0.058 |

TABLE IV
The relation between genotype distributions of
Leu125Val and other factors among CAD patients

| | Male | Female | Smoker | Nonsmoker | DM | Non-DM | HP | Non-HP |
|---|---|---|---|---|---|---|---|---|
| | (n = 112) | (n = 25) | (n = 53) | (n = 84) | (n = 87) | (n = 50) | (n = 83) | (n = 54) |
| CC(n) | 25 | 7 | 13 | 19 | 18 | 14 | 17 | 15 |
| CG(n) | 68 | 15 | 27 | 56 | 53 | 30 | 56 | 27 |
| GG(n) | 19 | 3 | 13 | 9 | 16 | 6 | 10 | 12 |
| p value | NS | | NS | | NS | | NS | |
| C(n) | 118 | 29 | 53 | 94 | 89 | 58 | 90 | 57 |
| G(n) | 106 | 21 | 53 | 74 | 85 | 42 | 76 | 51 |
| p value | NS | | NS | | NS | | NS | |

### Example 2

A PECAM-1-nil endotlielial-like cell line, Ren cells, were stably transfected with expression vector alone, wild type PECAM-1 cDNA constructs (Ren (+/WT), and PECAM-1 construct containing the combination of 125Val and 563Asn gene polymorphisms (Ren (+)/PM). By real time PCR, PECAM-1 gene expressions were measured and no difference found between Ren (+/WT) and Ren (+/PM) cells. However, by Western Blot assays, increased PECAM-1 levels in total cell lysate (1.4 folds), cytosol fraction (4 folds) containing small organelles and particles, and Triton X-100 (TX-100) insoluble fractions (2˜3 folds) of Ren (+/PM) cells were detected than that of Ren (+/WT). Moreover, ELISA uncovered an increase in soluble PECAM-1 (sPECAM-1) level (1.6 folds) in the culture medium of Ren (+/PM). In addition, enhanced cells aggregation (˜2.5 folds), and monocytes trans-Ren Cell migration (1.8 folds) were associated with Ren (+/PM) over Ren (+/WT).

An endothelial-like, PECAM-1 nil cell line, Ren cells, was stably transfected to over-express either human PECAM-1 gene of wild type or 125 Val and 536 Asn dual polymorphisms. In the later, high levels of cellular and soluble PECAM-1 protein were found, which is accompanied with increased Ren cell aggregation and monocytes trans-(Ren cells) migration. This study provided in vitro evidence of a patho-physiological role of PECAM-1 gene polymorphisms in promoting atherosclerosis and thrombosis. Platelet endothelial cell adhesion molecule-1 (PECAM-1) or CD31 is a 130-kDa membrane glycoprotein and a member of immunoglobulin (Ig) superfamily, an integral protein constitutively expressed and highly enriched at endothelial cell-cell junctions and on the surface of monocytes, some T-lymphocyte subsets, and platelets (37-40).

PECAM-1 has multiple functions including playing important roles in trans-endothelial migration of monocytes/leukocytes (40, 41) and also mediating cellular aggregation (42-44). As a trans-membrane glycoprotein, PECAM-1 has 6 (Ig)-like (homology) extracellular domains (encoded by exon 3 to 8), a short trans-membrane domain (encoded by exon 9) and a short cytoplasmic tail encoded by exon 10-16 (39, 45). The soluble form of PECAM-1 (sPECAM-1) has been detected in human plasma and in the medium of cultured endothelial cells (50-52) and elevated levels of sPECAM-1 in circulation have also been associated with AMI (52). sPECAM-1 was found to be increased in Chinese CAD patients and a link between Leu125Val gene polymorphism and sPECAM-1 levels (51) was also found.

Increased PECAM-1 protein levels were found in cytosol fractions and TX-100 insoluble fractions of Ren cells expressing the polymorphic PECAM-1. T his was accompanied with increased soluble PECAM-1, cell aggregation, and trans-(Ren Cell) migration of monocytes

### Cell Culture and Reagents

Ren cells, (a PECAM-1-null EC-like cell line, was kindly supplied by Dr. S M. Albelda, Department of Medicine, University of Pennsylvania School of Medicine). U-937 cell line was purchased from ATCC(CRL-1593.2, a human monocyte like, pre-monocyte lymphoma cell line) and employed in transmigration assay. RPMI1640 medium was used for the growth of both cell lines supplemented with 10% of fetal bovine serum, 1% penicillin/streptomycin, and 2 mM L-glutamine. REN cells transfected with human PECAM-1 were cultured in the same medium supplemented with selection reagent, G418 (50 μg/mL).

### Antibodies, Reagents and Chemicals

An anti-PECAM-1 monoclonal antibody was purchased from R&D Systems, Minneapolis, Minn., USA (clone 9G11, Cat# BBA7, raised against the extracellular domains of PECAM-1) was used in Western blot and immunofluorescence assays at 1:500 and 1:50, dilutions respectively. Soluble recombinant PECAM-1 (95˜98 KDa, containing only the extracellular domains) was also purchased from R&D Systems (Cat# ADP6C). G418 (neomycin, an antibiotics) was purchased from Sigma (G5013, 50 mg/mL). A polyclonal anti-PECAM-1 antibody, JHS-7 Ab, was prepared in our laboratory (55) and used as a blocking Ab in the aggregation assay. β-actin antibody (mouse anti-actin mAb, MAB 1501), use as 1:500 dilution for Western immunoblotting, was from CHEMICON International, Temecula, Calif.

Cloning of PECAM-1 cDNA Expression Constructs

Preparation of Wild Type PECAM-1 cDNA Construct

Wild type PECAM-1 cDNA was cloned from two expression sequencing tag (EST) of PECAM-1 purchased from Invitrogen. The entire wild type (WT) full length PECAM-1 cDNA sequence was inserted into an expression vector pIRESneo2 (Clonetech, Palo Alto, Calif.) containing the internal ribosome entry site (IRES) of the encephalomyocarditis virus (ECMV) promoter. WT PECAM-1 sequence was confirmed by sequencing.

Preparation of PECAM-1 cDNA Constructs with the Combination of SNPs (C+373G, Leu125Val and G+1688A (Ser563Asn) by Site-Directed Mutagenesis

Separated PECAM-1 cDNA constructs with the SNP of C+373G (Leu125Val) and G+1688A (Ser563Asn) were generated by site-directed mutagenesis using Pfu DNA Polymerase (Stratagene, La Jolla, Calif.) using wild type PECAM-1 construct as template. Single SNP PECAM-1 cDNA construct were confirmed by sequencing, then the combination of SNPs of C+373G (Leu125Val) and G+1688A (Ser563Asn) was generated by recombinant DNA technique (restriction enzyme Bam HI cut and paste). Finally this duel polymorphism construct was confirmed by sequencing.

### Establishment of REN Cells Lines Stably Expressing PECAM-1

Ren cell line was transfected with pIRESneo2 alone, named as Ren (−); with cDNA constructs of wild type PECAM-1, named as Ren (+/WT); and combined SNP (125Val plus 563Asn), named as Ren (+/PM), using a Effectin® transfection kit from Qiagen. Transfected cells were subjected to G418 (50 ug/L). Selection after 48 hours culture in RPMI medium. Finally, transfected Ren cells lines were characterized by RT-PCR and immunofluorescent assay and Western Blot assays with a mAb against human PECAM-1.

### PECAM-1 Gene Transcription Assay

Total RNA was isolated from Ren cells with Trizol Reagent® (Invitrogen) following the manufacturer's instructions. After DNase I (RNase free, Roche) treatment, first-strand complementary DNA (cDNA) was synthesized by reverse transcription (RT) primed with both an oligo d (T) 15 and a random hexamer using a standard protocol. Real time polymerase chain reactions (real time PCR) were prepared using SYBR® GREEN PCR MASTER MIX (Cat#4309155) from Applied Biosystems, Warrington, UK. The PCRs were carried out on iCycler™ IQ (real time PCR machine from Bio-Red). The sequences of PCR primers (listed as Forward primers (5′-3′) and Reverse primers (5′-3′) followed by the sizes of PCR products (number of base pairs) are: PECAM-1: GCTGACCCTTCTGCTCTGTT and TGAGAGGTGGTGCTGACATC (150); and β-actin: CATGTACGTTGCTATCCAGGC and CTCCTTAATGTCACGCACGAT (250). Another pair of PECAM-1 for normal RT-PCR is as follows: CCCGAACTGGAATCTTCCTT and GGGTTTGCCCTCTTTTTCTC (651). β-actin gene served as an internal control. PCRs were performed by repeating the following amplification cycle 32 times: denaturing at 94° C. for 45 seconds followed by annealing at 60° C. for 45 seconds and extension at 72° C. for 60 seconds. Results from the real time PCR were presented as threshold cycles normalized to that of β-actin gene according to the method of Overbergh, et al (20). It reversibly represented the concentration (number of copies) of cDNA template of individual gene.

### Detecting PECAM-1 in Membrane and Cytosol Fractions

Membrane and cytosolic fractions of Ren cells were prepared according to Li, et. al. (57). Briefly, Ren cells (˜2×10⁶) were harvested on ice and sonicated for three times (15 seconds each time). Nuclei and unbroken cells in lysate were removed by centrifugation at 1475×g for 15 min at 4° C. and the supernatant was centrifuged for 15 min at 29,000×g to obtain a pellet of large subcellular organelles (Cytosol L). Next, the supernatant of above fraction was centrifuged for 15 min at 29,000×g to obtain a pellet containing submitochrondrial particles, smaller organelles, and some microsomes (Cytosol S). Finally, the supernatant was centrifuged at 120,000×g for 30 min to obtain a pellet of membrane fraction (Membrane).

### Preparation of Detergent-Soluble and Insoluble (Cytoskeleton) Fractions

Triton X-100 soluble and insoluble fractions were prepared from cell lysate (˜2×10⁶ Ren cells) according to Amos et al (58). The purity of detergent soluble/insoluble fractions was verified by detecting the presence of intercellular cell adhesion molecule-1 (ICAM-1) using Western blot assay (data not shown) as ICAM-1 is predominantly presents in the soluble fraction only (58).

### Measurement of PECAM-1 Protein by Western Blot Assay

Ren cells were lysed by M-PERTM mammalian protein extraction reagent (Pierce Biotechnology Inc., Rockford, Ill.) with the addition of 10% Complete TM EDTA-free Protease Inhibitor Cocktail (Roche). Equal amounts (25 μg) of total protein from total cell lysates, cytosolic or membrane fractions, Triton X-100 soluble/insoluble fractions were subjected to 10% sodium dodecyl sulfate-polyacrylamide gel electrophoresis (SDSPAGE) and Western immunoblotting assay probed by primary mAb of PECAM-1 followed by secondary rabbit IgG. Finally the blots were visualized by enhanced chemiluminescence. All blots were stripped and re-probed with anti-β-actin antibody. Protein quantifications were performed with densitometry scanning of the Western Blot images of both PECAM-1 and β-actin and the PECAM-1 quantifications were conducted by standardization against β-actin.

### Immunofluorescent Confocal Microscopy

Cells were grown on glass coverslips until confluence. Then cells were fixed in 4% formaldehyde in PBS for 15 minutes at room temperature and subsequently permeabilized in 0.2% Triton X-100 in PBS for 30 minutes. Then the cells were incubated with primary anti-PECAM-1 mAb for 1 hour followed by secondary FITC conjugated anti-rabbit IgG (1:300)—for 1 hour at room temperature. The fluorescent images were viewed using confocal microscopy.

### Measurement of Soluble PECAM-1 Protein in Cell Culture Medium by ELISA

Ren cell culture medium was collected from confluent cell culture and immediately centrifuged at 5,000 rpm for 5 minutes and the supernatant was filtered and concentrated (3:1) with a filter YM-10 (10 KDa MW) from Millipore, Bedford, Mass. Indirect enzyme linked immunosorbent assay (Sandwich ELISA, BMS229, Bender MedSystem, Vienna, Austria) was used for quantitative detection of the levels of sPECAM-1.

### Transmigration Assay

Leukocyte trans-(Ren cell) migration ™ assays were performed on a Transwell® design (A plate of 12 wells equipped with insert upper wells with 3-μmicron pore size polycarbonate membrane bottom from Costar, Cambridge, Mass.) according to a protocol described previously (55). In brief, Ren cells (105 cells) were seeded on gelatin (0.2%)-coated upper wells and allowed to grow for 2 days until confluence (This could be determined by fluorescent staining with of cells grown in additional wells using cell tracker dye from Molecular Probes). Next, 106 U-937 cells were added in each upper well and allowed to transmigrate through Ren cells monolayers for 12 hours at 37° C. with 5% CO₂. Finally, U-937 cells migrated to the bottom wells were collected and counted.

### Cell Aggregation Assay

A variation of aggregation assay of Takeichi (59) was adopted. Briefly, confluent culture of Ren cells were detached and dispersed and resuspended to about 106 cells/ml in HBSS (37° C.) with 1 mM CaCl2. One ml cell aliquots were transfected to wells in a 24-well plate and rotated on a gyratory shaker (At 90 rpm) at 37° C. for 20 minutes, then stopped by addition of glutaraldehyde to a final concentration of 2%. Aggregation was quantified by examining representative aliquot of equal volumes from each sample on a hemacytometer grid under phase contrast optics. Cells remaining as single cells or present in aggregates (≧3 cells clots) were counted in nine squares. Data are expressed as the percent of total cells present in aggregates.

### Statistics

All statistical tests were calculated from three or more independent experiments. Comparisons were made by paired t test, and a p value less than 0.05 was considered statistically significant.

### Characterization of PECAM-1 Gene/Protein Expression in Transfected Ren Cells

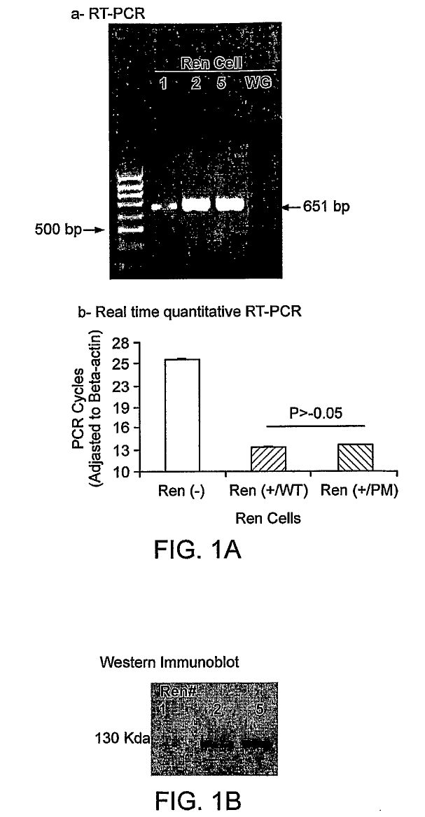

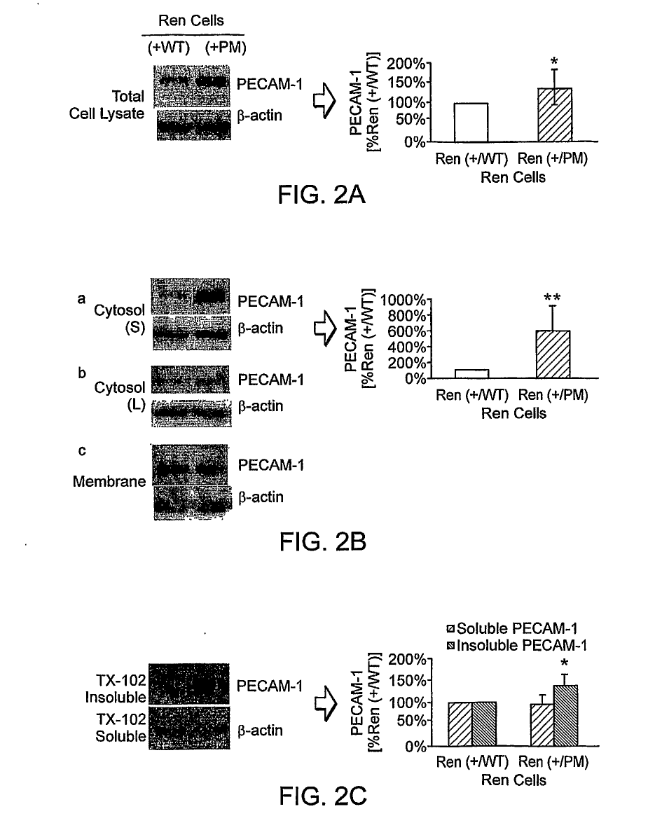

Abundant PECAM-1 gene and protein expression in PECAM-1 transfected Ren cells, Ren (+/WT) and Ren (+/PM), were observed (FIG. 1). PECAM-1 gene expression in transfected Ren cells was well characterized by RT-PCR and measured by real time quantitative RT-PCR (FIG. 1A). PECAM-1 protein mass was determined by Western Immunoblot (FIG. 1B). Although residual PECAM-1 gene transcription in Ren (−) cells was noted by over-amplification of the cDNA by RT-PCR (40 cycles) (FIG. 1Aa), no PECAM-1 protein was detected in Ren (−) by Western blot (FIG. 1B) and IF (FIG. 2D).

### PECAM-1 Protein Expression and Distribution in Transfected Ren Cells

Although real-time RT-PCR assays failed to show any differences in PECAM-1 gene transcription between two Ren cell lines expressing PECAM-1 (FIG. 1A), increased PECAM-1 protein levels were observed in total cell lysates (1.4 fold) in Ren (+/PM) in comparison with that in Ren (+/WT) by both Western Blot (FIG. 2A). Moreover, more significant increase in PECAM-1 levels was found in sub-cellular fractions including a cytosolic fraction containing small organelles and particles (FIG. 2Ba) and Triton X-100 insoluble fraction (FIG. 2C) derived from Ren (+/PM). All PECAM-1 detected by Western Blots has a molecular weight of 130 KDa. Confocol immunofluorescence analysis with anti-PECAM-1 antibody suggested an increase in PECAM-1 mass in Ren (+/PM) cells in general (FIG. 2D), also enhanced sub-cellular staining adjacent to membrane was observed which may reflect the increased in cytosolic PECAM-1.

The Levels of sPECAM-1 in Ren Cell Culture Medium

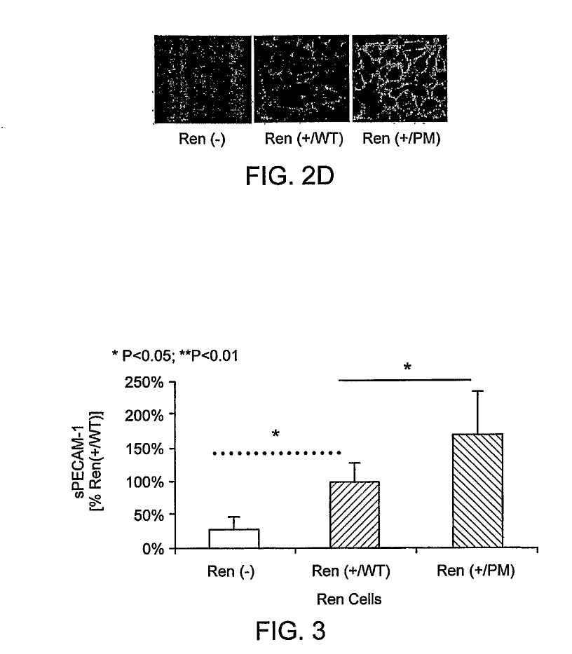

To correlate our finding of elevated sPECAM-1 levels in the plasma of CAD patients (both Chinese and Indians), levels of sPECAM-1 in Ren cell culture medium was measured with the identical ELISA kit. As expected, the levels of sPECAM1 in the culture medium of transfected Ren cells was about 4 fold higher than that of Ren (−) cells. However, sPECAM-1 measured in the culture medium of Ren (+/PM) cells was found about 2 folds of that Ren (+/WT) cells (FIG. 3).

### Ren Cell Aggregation ASSAYS

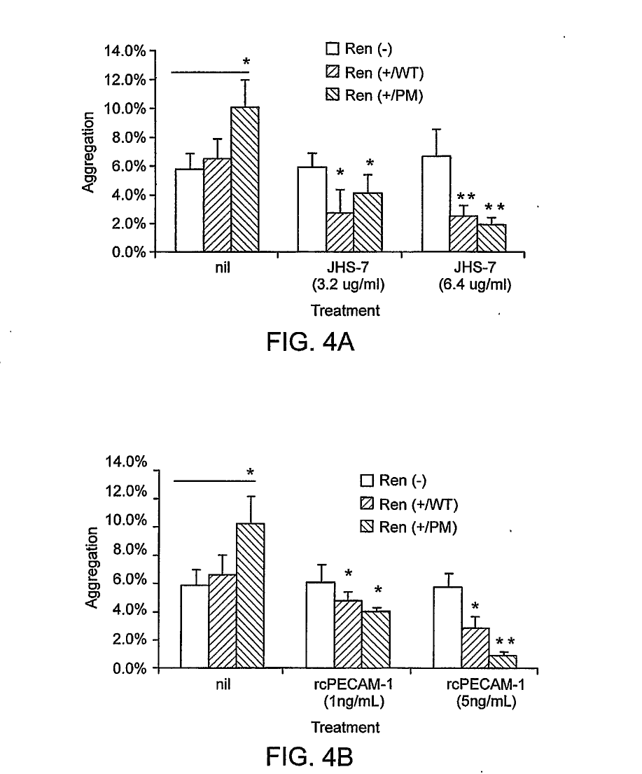

Ren cell aggregation assay was conducted to mimic platelet aggregation in vivo. As shown in FIG. 4, a ˜1.5 fold increase in Ren cell aggregation was observed in Ren (+/PM) as compared with Ren (+/WT) while slightly increased aggregation was found in Ren (+/WT) than Ren (−). The addition of a PECAM-1 antibody and a recombinant human PECAM-1 protein (containing the entire extracellular Ig-like domains of PECAM-1) attenuated the aggregation in both PECAM-1 transfected Ren cell lines [Ren (+/WT) and Ren (+/PM)], but not PECAM-1 negative Ren cells [Ren (−)].

### U-937 Cell Trans-(Ren-Cell) Migration Assays

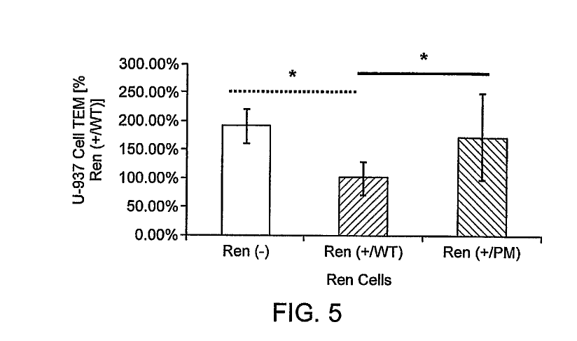

Monolayers of Ren cell, were subjected to U-937 cells trans-migration assay, an experiment to mimic leukocyte diapedesis in vivo. As shown in FIG. 5, the trans-(Ren cell monolayer) migration of U-937 cells was found much lower in Ren (+/WT) as compared with Ren (−) firstly. However, a ˜1.8 folds increase in U-937 cells transmigration was observed in Ren (+/PM) on top of Ren (+/WT).

PECAM-1 is a major constitutively expressed protein in mammalian endothelial cells and has been implicated in endothelial inflammation/dysfunction (60, 61). The fully expressed PECAM-1 protein (130 kDa) and a soluble PECAM-1 (110 kDa) have been detected in human cells (37, 62), and plasma respectively (52, 62).

REN transfected with a polymorphic PECAM-1 bearing the combination of 125 Val, and 563Asn, i.e. Ren (+/PM) increased PECAM-1 in detergent insoluble fraction and increased sPECAM-1 without affecting gene expression. Ren (+/PM) promoted cell aggregation and monocyte transmigration, indicating functional effects of PECAM-1 gene polymorphisms on cell aggregation and monocyte transmigration. PECAM-1 mediates cellular aggregation (42-44) via homophilic binding via its 1st and 2nd extracellular (Ig)-like domain (38, 41, 46, 48, 63-66). Elevated aggregation found in Ren cells expressing polymorphic PECAM-1 gene suggested that such polymorphisms are prone to platelet aggregation and thrombogenesis. PECAM-1 has been associated with inter-endothelial adhesion and leukocyte-endothelial interactions, particularly during transmigration (47, 67). Such leukocyte recruiting to endothelium involves PECAM-1 homophilic binding. In this study, employing endothelial-like Ren cells, increased transmigration of U-937 cells through the monolayer of Ren (+/PM) was found in comparison with Ren (+/WT), which suggested a potential role of PECAM-1 SNPs on the permeability of endothelium. Such PECAM-1 gene polymorphism may render endothelium vulnerable to inflammatory challenges and prone to atherogenesis.

The expression of wild type PECAM-1 in Ren cells (Ren (+/WT)) seems to have a negative impact on TEM compared with PECAM-1 nil Ren cells (Ren (−)). A similar finding has been reported in which cultured PECAM-KO endothelial cells exhibit prolonged permeability changes in response to histamine treatment compared with PECAM-1-reconstituted endothelial cells (68). Thus recent understanding on PECAM-1 has its role on maintaining the endothelium integrality from inflammatory and injury stimuli (69). It is proposed that PECAM-1 acts as an inhibitor of cellular activation via protein tyrosine kinase-dependent signaling pathways as it serves as an inhibitory receptor that modulates platelet responses to collagen (70, 71).

Homozygous 125Val of PECAM-1 was associated with a ˜2 folds increase of sPECAM-1 in plasma compared to homozygous of 125Leu allele plus heterozygous of 125Leu/Val. Higher sPECAM-1 (1.8 folds) was found in the culture medium of Ren (+/PM), which expresses the cDNA of PECAM-1 carrying combined 125Val and 563Asn polymorphisms compared to that of Ren cells expressing WT PECAM-1. Thus, there is a correlation between genotype of PECAM-1 and the sPECAM-1 levels.

Ren cells have been used for studying endothelial cells (72, 73) and PECAM-1 expression in transfected Ren cells resembles that in HUVECs. PECAM-1 null cell line, Ren cells, was stably transfected and over-expressing human PECAM-1 gene of wild type and 125Val and 536Asn dual polymorphisms.

Expression of PECAM-1 combined polymorphisms 125Val and 563Asn in Ren cells results in an increase in detergent fraction of PECAM-1 as well as sPECAM-1 in the culture medium, which is accompanied by increased Ren cell aggregation and monocytes trans-(Ren cells) migration. Increased shedding of sPECAM-1 in cell culture medium corresponds to increased plasma sPECAM-1 in CAD patients.

Histochemical detection of nuclearapoptotic bodies by DAPI. Morphologic changes in the nuclear chromatin of cells undergoing apoptosis are detected by staining with the DNA-binding fluorochrome (DAPI). Cells (5×10³) are grown on sterilized glass coverslips in six-well plastic tissue culture dishes (Falcon multiwell, Becton-Dickinson Labware, Franklin Lakes, N.J.). After 24 h, remove medium and add fresh serum-free medium along with C-2 ceramide [10 μM in dimethyl sulfoxide (DMSO)] to serve as a positive control, and C-2 dihydroceramide (10 μM in DMSO) is added as a negative control. Next, various GSL (solubilized in DMSO) are added. Vehicle control (DMSO) not to exceed 0.01% of the medium volume is also added. After a 24-h incubation period, cells are washed twice with PBS and incubated in 30% acetic acid in methanol for 3 min at room temperature. The fixing solution is removed, and cells are washed twice in PBS. Next ˜20 μl of a solution is added and incubated for 5 min at room temperature. Alternatively, after fixing, the cells can be stored at −20° for several months, brought to room temperature, and stained as described earlier and transferred to a glass slide. Next 500 cells per slide are scored for the incidence of apoptotic chromatin changes. The slides are viewed under a fluorescence microscope. Cells with three or more chromatin fragments are considered apoptotic.

### Angiogenesis Assay

Procure Angiogenesis Assay Kit from CHEMICON Inc., Cat # ECM 630:

1. Thaw the diluent buffer provided with the kit in ice.

2. Add 100 μL of diluent buffer to the vial of matrix solution and mix gently. Its necessary to cut the edge of the tip so that air bubbles will not be introduced also this will aid in smooth pipetting of viscous matrix solution.

3. Transfer 50 μL of diluted matrix solution on to each well of 96 well tissue culture plate. Take care not to introduce air-bubbles.

4. Incubate 37° C. carbon dioxide incubator for 1-2 hrs to allow solidification of the matrix.

5. Treat endothelial cells or endothelial like cells with various agonists/antagonists. Harvest them and resuspend in medium containing 2% serum. It's important to have the agonists/antagonists in the suspension at this stage also.

6. Seed 5×10³-1×10⁴ cells per well (˜10-12 μL from 25 mm culture dish or from a well of six well culture plate).

7. Incubate at 37° C. carbon dioxide incubator overnight. Typically tube formation can be seen after 6 hrs of incubation and can be documented at 8-12 hrs. After 20 hrs the tubes will get distorted as cells undergo apoptosis.

8. Documentation: Photograph 5-6 fields/well at 10×. Then count number of tubes, i.e. lines connecting each cell. Then average number of tubes and express as it as No of Tubes/well.
9. Experiments should be done in triplicates per agonist/antagonist treatment.

### REFERENCES

1. Ross R. Atherosclerosis—an inflammatory disease. N Engl J Med. 1999; 340:115-26.
2. Davies M J, Gordon J L, Gearing A J, Pigott R, Woolf N, Katz D, Kyriakopoulos A. The expression of the adhesion molecules ICAM-1, VCAM-1, PECAM, and E-selectin in human atherosclerosis. J Pathol. 1993; 171:223-9.
3. Price D T, Loscalzo J. Cellular adhesion molecules and atherogenesis. Am J Med. 1999; 107:85-97.
4. Huo Y, Ley K. Adhesion molecules and atherogenesis. Acta Physiol Scand. 2001; 173:35-43.
5. Ohto H, Maeda H, Shibata Y, Chen R F, Ozaki Y, Higashihara M, Takeuchi A, Tohyama H. A novel leukocyte differentiation antigen: two monoclonal antibodies TM2 and TM3 define a 120-kd molecule present on neutrophils, monocytes, platelets, and activated lymphoblasts. Blood. 1985; 66:873-81.
6. Newman P J, Berndt M C, Gorski J, White G C, 2nd, Lyman S, Paddock C, Muller W A. PECAM-1 (CD31) cloning and relation to adhesion molecules of the immunoglobulin gene superfamily. Science. 1990; 247:1219-22.
7. Albelda S M, Oliver P D, Romer L H, Buck C A. EndoCAM: a novel endothelial cell-cell adhesion molecule. J Cell Biol. 1990; 110:1227-37.
8. Yan H C, Pilewski J M, Zhang Q, DeLisser H M, Romer L, Albelda S M. Localization of multiple functional domains on human PECAM-1 (CD31) by monoclonal antibody epitope mapping. Cell Adhes Commun. 1995; 3:45-66.
9. Kirschbaum N E, Gumina R J, Newman P J. Organization of the gene for human platelet/endothelial cell adhesion molecule-1 shows alternatively spliced isoforms and a functionally complex cytoplasmic domain. Blood. 1994; 84:4028-37.
10. Muller W A, Weigl S A, Deng X, Phillips D M. PECAM-1 is required for transendothelial migration of leukocytes. J Exp Med. 1993; 178:449-60.
11. Sun J, Williams J, Yan H C, Amin K M, Albelda S M, DeLisser H M. Platelet endothelial cell adhesion molecule-1 (PECAM-1) homophilic adhesion is mediated by immunoglobulin-like domains 1 and 2 and depends on the cytoplasmic domain and the level of surface expression. J Biol Chem. 1996; 271:18561-70.
12. Matsumura T, Wolff K, Petzelbauer P. Endothelial cell tube formation depends on cadherin 5 and CD31 interactions with filamentous actin. J Immunol. 1997; 158:3408-16.
13. Piali L, Hammel P, Uherek C, Bachmann F, Gisler R H, Dunon D, Imhof B A. CD31/PECAM-1 is a ligand for alpha v beta 3 integrin involved in adhesion of leukocytes to endothelium. J Cell Biol. 1995; 130:451-60.
14. Gao C, Sun W, Christofidou-Solomidou M, Sawada M, Newman D K, Bergom C, Albelda S M, Matsuyama S, Newman P J. PECAM-1 functions as a specific and potent inhibitor of mitochondrial-dependent apoptosis. Blood. 2003; 102:169-79.
15. Vaporciyan A A, DeLisser H M, Yan H C, Mendiguren, II, Thom S R, Jones M L, Ward P A, Albelda S M. Involvement of platelet-endothelial cell adhesion molecule-1 in neutrophil recruitment in vivo. Science. 1993; 262:1580-2.
16. Mahooti S, Graesser D, Patil S, Newman P, Duncan G, Mak T, Madri J A. PECAM-1 (CD31) expression modulates bleeding time in vivo. Am J Pathol. 2000; 157:75-81.
17. Wenzel K, Baumann G, Felix S B. The homozygous combination of Leu125Val and Ser563Asn polymorphisms in the PECAM1 (CD31) gene is associated with early severe coronary heart disease. Hum Mutat. 1999; 14:545.
18. Sasaoka T, Kimura A, Hohta S A, Fukuda N, Kurosawa T, Izumi T. Polymorphisms in the platelet-endotlielial cell adhesion molecule-1 (PECAM-1) gene, Asn563Ser and Gly670Arg, associated with myocardial infarction in the Japanese. Ann NY Acad Sci. 2001; 947:259-69; discussion 269-70.
19. Gurubhagavatula I, Amrani Y, Pratico D, Ruberg F L, Albelda S M, Panettieri R A, Jr. Engagement of human PECAM-1 (CD31) on human endothelial cells increases intracellular calcium ion concentration and stimulates prostacyclin release. J Clin Invest. 1998; 101:212-22.
20. Serebruany V L, Murugesan S R, Pothula A, Semaan H, Gurbel P A. Soluble PECAM-1, but not P-selectin, nor osteonectin identify acute myocardial infarction in patients presenting with chest pain. Cardiology. 1999; 91:50-5.
21. Goldberger A, Middleton K A, Oliver J A, Paddock C, Yan H C, DeLisser H M, Albelda S M, Newman P J. Biosynthesis and processing of the cell adhesion molecule PECAM-1 includes production of a soluble form. J Biol Chem. 1994; 269:17183-91.
22. Blin N, Stafford D W. A general method for isolation of high molecular weight DNA from eukaryotes. Nucleic Acids Res. 1976; 3:2303-8.
23. Behar E, Chao N J, Hiraki D D, Krishnaswamy S, Brown B W, Zehnder J L, Grumet F C. Polymorphism of adhesion molecule CD31 and its role in acute graft-versus-host disease. N Engl J Med. 1996; 334:286-91.
24. Maruya E, Saji H, Seki S, Fujii Y, Kato K, Kai S, Hiraoka A, Kawa K, Hoshi Y, Ito K, Yokoyama S, Juji T. Evidence that CD31, CD49b, and CD62L are immunodominant minor histocompatibility antigens in HLA identical sibling bone marrow transplants. Blood. 1998; 92:2169-76.
25. Serebruany V L, Gurbel P A. Effect of thrombolytic therapy on platelet expression and plasma concentration of PECAM-1 (CD31) in patients with acute myocardial infarction. Arterioscler Thromb Vasc Biol. 1999; 19:153-8.
26. Sanger F, Nicklen S, Coulson A R. DNA sequencing with chain-terminating inhibitors. Proc Natl Acad Sci USA. 1977; 74:5463-7.
27. Staden R. Automation of the computer handling of gel reading data produced by the shotgun method of DNA sequencing. Nucleic Acids Res. 1982; 10:4731-51.
28. Bachorik P S. Measurement of total cholesterol, HDL-cholesterol, and LDL-cholesterol. Clin Lab Med. 1989; 9:61-72.
29. Gardemann A, Knapp A, Katz N, Tillmanns H, Haberbosch W. No evidence for the CD31 C/G gene polymorphism as an independent risk factor of coronary heart disease. Thromb Haemostat. 2000; 83:629.
30. Liao F, Huynh H K, Eiroa A, Greene T, Polizzi E, Muller W A. Migration of monocytes across endothelium and passage through extracellular matrix involve separate molecular domains of PECAM-1. J Exp Med. 1995; 182:1337-43.
31. Enas E A, Garg A, Davidson M A, Nair V M, Huet B A, Yusuf S. Coronary heart disease and its risk factors in first-generation immigrant Asian Indians to the United States of America. Indian Heart J. 1996; 48:343-53.
32. Stein C E, Fall C H, Kumaran K, Osmond C, Cox V, Barker D J. Fetal growth and coronary heart disease in south India. Lancet. 1996; 348:1269-73.
33. Blann A D, Wadley M S, Dobrotova M, Sanders P, Jayson M I, McCollum C N. Soluble platelet endothelial cell adhesion molecule-1 (sPECAM-1) in inflammatory vascular disease, atherosclerotic vascular disease, and in cancer. Blood Coagul Fibrinolysis. 1998; 9:99-103.
34. Cockerill G W, Rye K A, Gamble J R, Vadas M A, Barter P J. High-density lipoproteins inhibit cytokine-induced expression of endothelial cell adhesion molecules. Arterioscler Thromb Vasc Biol. 1995; 15:1987-94.
35. Haller H, Schaper D, Ziegler W, Philipp S, Kuhlmann M, Distler A, Luft F C. Low-density lipoprotein induces vascular adhesion molecule expression on human endothelial cells. Hypertension. 1995; 25:511-6.
36. Vora D K, Fang Z T, Liva S M, Tyner T R, Parhami F, Watson A D, Drake T A, Territo M C, Berliner J A. Induction of P-selectin by oxidized lipoproteins. Separate effects on synthesis and surface expression. Circ Res. 1997; 80:810-8.
37. Newman P J, Berndt M C, Gorski J, White G C, 2nd, Lyman S, Paddock C, et al. PECAM-1 (CD31) cloning and relation to adhesion molecules of the immunoglobulin gene superfamily. Science 1990; 247(4947):1219-22.
38. DeLisser H M, Newman P J, Albelda S M. Platelet endothelial cell adhesion molecule (CD31). Curr Top Microbiol Immunol 1993; 184:37-45.
39. Kirschbaum N E, Gumina R J, Newman P J. Organization of the gene for human platelet/endothelial cell adhesion molecule-1 shows alternatively spliced isoforms and a functionally complex cytoplasmic domain. Blood 1994; 84(12):4028-37.
40. Newman P J. The biology of PECAM-1. J Clin Invest 1997; 99(1):3-8.
41. Muller W A, Weigl S A, Deng X, Phillips D M. PECAM-1 is required for transendothelial migration of leukocytes. J Exp Med 1993; 178(2):449-60.
42. Rosenblum W I, Nelson G H, Wormley B, Werner P, Wang J, Shih C C. Role of platelet-endothelial cell adhesion molecule (PECAM) in platelet adhesion/aggregation over injured but not denuded endothelium in vivo and ex vivo. Stroke 1996; 27(4):709-11.
43. DeLisser H M, Yan H C, Newman P J, Muller W A, Buck C A, Albelda S M. Platelet/endothelial cell adhesion molecule-1 (CD31)-mediated cellular aggregation involves cell surface glycosaminoglycans. J Biol Chem 1993; 268(21):16037-46.
44. Xie Y, Muller W A. Molecular cloning and adhesive properties of murine platelet/endotlielial cell adhesion molecule 1. Proc Natl Acad Sci USA 1993; 90(12):5569-73.
45. Yan H C, Pilewski J M, Zhang Q, DeLisser H M, Romer L, Albelda S M. Localization of multiple functional domains on human PECAM-1 (CD31) by monoclonal antibody epitope mapping. Cell Adhes Commun 1995; 3(1):45-66.
46. Thompson R D, Noble K E, Larbi K Y, Dewar A, Duncan G S, Mak T W, et al. Platelet-endothelial cell adhesion molecule-1 (PECAM-1)-deficient mice demonstrate a transient and cytokine-specific role for PECAM-1 in leukocyte migration through the perivascular basement membrane. Blood 2001; 97(6):1854-60.
47. Newton J P, Buckley C D, Jones E Y, Simmons D L. Residues on both faces of the first immunoglobulin fold contribute to homophilic binding sites of PECAM-1/CD31. J Biol Chem 1997; 272(33):20555-63.
48. Sun Q H, DeLisser H M, Zukowski M M, Paddock C, Albelda S M, Newman P J. Individually distinct Ig homology domains in PECAM-1 regulate homophilic binding and modulate receptor affinity. J Biol Chem 1996; 271(19):11090-8.
49. Wenzel K, Baumann G, Felix S B. The homozygous combination of Leu125Val and Ser563Asn polymorphisms in the PECAM1 (CD31) gene is associated with early severe coronary heart disease. Hum Mutat 1999; 14(6):545.
50. Sasaoka T, Kimura A, Hohta S A, Fukuda N, Kurosawa T, Izumi T. Polymorphisms in the platelet-endothelial cell adhesion molecule-1 (PECAM-1) gene, Asn563Ser and Gly670Arg, associated with myocardial infarction in the Japanese. Ann N Y Acad Sci 2001; 947:259-69; discussion 269-70.
51. Wei H, Fang L, Chowdhury S H, Gong N, Xiong Z, Song J, et al. Plateletendothelial cell adhesion molecule-1 gene polymorphism and its soluble level are associated with severe coronary artery stenosis in Chinese Singaporean. Clin Biochem 2004; 37(12):1091-7.
52. Serebruany V L, Murugesan S R, Pothula A, Semaan H, Gurbel P A. Soluble PECAM-1, but not P-selectin, nor osteonectin identify acute myocardial infarction in patients presenting with chest pain. Cardiology 1999; 91(1):50-5.
53. Elrayess M A, Webb K E, Bellingan G J, Whittall R A, Kabir J, Hawe E, et al. R643G polymorphism in PECAM-1 influences transendothelial migration of monocytes and is associated with progression of CHD and CHD events. Atherosclerosis 2004; 177(1):127-35.
54. Gurubhagavatula I, Amrani Y, Pratico D, Ruberg F L, Albelda S M, Panettieri R A, Jr. Engagement of human PECAM-1 (CD31) on human endothelial cells increases intracellular calcium ion concentration and stimulates prostacyclin release. J Clin Invest 1998; 101(1):212-22.
55. Wei H, Song J, Fang L, Li G, Chatterjee S. Identification of a novel transcript of human PECAM-1 and its role in the transendothelial migration of monocytes and Ca2+ mobilization. Biochem Biophys Res Commun 2004; 320(4):1228-35.
56. Overbergh L, Valckx D, Waer M, Mathieu C. Quantification of murine cytokine mRNAs using real time quantitative reverse transcriptase PCR. Cytokine 1999; 11(4):305-12.
57. Li J M, Shah A M. Intracellular localization and preassembly of the NADPH oxidase complex in cultured endothelial cells. J Biol Chem 2002; 277(22):19952-60.
58. Amos C, Romero I A, Schultze C, Rousell J, Pearson J D, Greenwood J, et al. Cross-linking of brain endothelial intercellular adhesion molecule (ICAM)-1 induces association of ICAM-1 with detergent-insoluble cytoskeletal fraction. Arterioscler Thromb Vasc Biol 2001; 21(5):810-6.
59. Takeichi M. Functional correlation between cell adhesive properties and some cell surface proteins. J Cell Biol 1977; 75(2 Pt 1):464-74.
60. Vaporciyan A A, DeLisser H M, Yan H C, Mendiguren, II, Thom S R, Jones M L, et al. Involvement of platelet-endothelial cell adhesion molecule-1 in neutrophil recruitment in vivo. Science 1993; 262(5139): 1580-2.
61. Albelda S M, Lau K C, Chien P, Huang Z Y, Arguiris E, Bohen A, et al., Role for platelet-endothelial cell adhesion molecule-1 in macrophage Fcgamma receptor function. Am J Respir Cell Mol Biol 2004; 31(2):246-55.
62. Goldberger A, Middleton K A, Oliver J A, Paddock C, Yan H C, DeLisser H M, et al. Biosynthesis and processing of the cell adhesion molecule PECAM-1 includes production of a soluble form. J Biol Chem 1994; 269(25):17183-91.
63. Muller W A. The role of PECAM-1 (CD31) in leukocyte emigration: studies in vitro and in vivo. J Leukoc Biol 1995; 57(4):523-8.
64. Wakelin M W, Sanz M J, Dewar A, Albelda S M, Larkin S W, Boughton-Smith N, et al. An anti-platelet-endothelial cell adhesion molecule-1 antibody inhibits leukocyte extravasation from mesenteric microvessels in vivo by blocking the passage through the basement membrane. J Exp Med 1996; 184(1):229-39.
65. Bogen S, Pak J, Garifallou M, Deng X, Muller W A. Monoclonal antibody to murine PECAM-1 (CD31) blocks acute inflammation in vivo. J Exp Med 1994; 179(3):1059-64.
66. Liao F, Huynh H K, Eiroa A, Greene T, Polizzi E, Muller W A. Migration of monocytes across endothelium and passage through extracellular matrix involve separate molecular domains of PECAM-1. J Exp Med 1995; 182(5):1337-43.
67. Nakada M T, Amin K, Christofidou-Solomidou M, O'Brien C D, Sun J, Gurubhagavatula I, et al. Antibodies against the first Ig-like domain of human platelet endothelial cell adhesion molecule-1 (PECAM-1) that inhibit PECAM-1-dependent homophilic adhesion block in vivo neutrophil recruitment. J Immunol 2000; 164(1):452-62.
68. Graesser D, Solowiej A, Bruckner M, Osterweil E, Juedes A, Davis S, et al. Altered vascular permeability and early onset of experimental autoimmune encephalomyelitis in PECAM-1-deficient mice. J Clin Invest 2002; 109(3):383-92.
69. Newman P J, Newman D K. Signal transduction pathways mediated by PECAM-1: new roles for an old molecule in platelet and vascular cell biology. Arterioscler Thromb Vasc Biol 2003; 23(6):953-64.
70. Jones K L, Hughan S C, Dopheide S M, Farndale R W, Jackson S P, Jackson D E. Platelet endothelial cell adhesion molecule-1 is a negative regulator of platelet-collagen interactions. Blood 2001; 98(5):1456-63.
71. Zibara K, Chignier E, Covacho C, Poston R, Canard G, Hardy P, et al. Modulation of expression of endothelial intercellular adhesion molecule-1, plateletendothelial cell adhesion molecule-1, and vascular cell adhesion molecule-1 in aortic arch lesions of apolipoprotein E-deficient compared with wild-type mice. Arterioscler Thromb Vasc Biol 2000; 20(10):2288-96.
72. O'Brien C D, Lim P, Sun J, Albelda S M. PECAM-1-dependent neutrophil transmigration is independent of monolayer PECAM-1 signaling or localization. Blood 2003; 101(7):2816-25.
73. Ji G, O'Brien C D, Feldman M, Manevich Y, Lim P, Sun J, et al. PECAM-1 (CD31) regulates a hydrogen peroxide-activated nonselective cation channel in endothelial cells. J Cell Biol 2002; 157(1):173-84.

The references referred to herein are each incorporated by reference in their entirety. A number of embodiments of the invention have been described. Nevertheless, it will be understood that various modifications may be made without departing from the spirit and scope of the invention. Accordingly, other embodiments are within the scope of the following claims.

1. A method of assessing risk of artherosclerotic disease in a subject comprising:
determining a PECAM-1 genotype status of a subject, and
correlating the genotype status to a subject's risk of developing artherosclerotic disease.

2. The method of claim 1, further comprising correlating the genotype status to a therapeutic treatment.

3. The method of claim 1, wherein the genotype status is determined by one or more of immunological methods or sequencing methods.

4. The method of claim 1, wherein the PECAM-1 genotype status at one or more of amino acid positions 125 or 563 are determined.

5. The method of claim 1, wherein the PECAM-1 genotype status at one or more nucleotide positions 373 or 1688 are determined.

6. The method of claim 5, wherein the PECAM-1 genotype status is determined by PCR methods, immunological methods, sequencing methods, expression level of PECAM-1, level of soluble PECAM-1, enzyme kinetics of PECAM-1, SNP Chip technology, RFLP, gean function assays (such as adhesion, trans-endothelial migration and angiogenesis).

7-19. (canceled)

20. A method of selecting a subject for treatment of an artherosclerotic disease, comprising:
detecting the presence or absence of a variation at one or more of amino acid position 432, nucleotide position 373, amino acid position 563, or nucleotide position 1688 of PECAM-1, and
correlating an presence of a variation or heterozygous variation with an indication of increased risk of artherosclerotic disease.

21. The method of claim 20, further comprising correlating the absence of a variation with an indication of decreases risk that a subject will develop artherosclerotic disease.

22. The method of claim 20, wherein the detecting comprises PCR methods, immunological methods, sequencing methods, expression level of PECAM-1 gene, expression level of PECAM-1 protein, and enzyme kinetics of PECAM-1.

23. (canceled)

24. A method for determining the therapeutic capacity of a candidate anti-artherosclerotic agent in a subject, comprising:
determining a PECAM-1 genotype status of a subject or a cell of a subject;
determining a pre-treatment artherosclerotic disease status in the subject;
administering a therapeutically effective amount of a candidate anti-artherosclerotic agent to the subject; and
determining a post-treatment artherosclerotic disease status in the subject.

25. The method of claim 24, wherein a modulation of artherosclerotic disease status indicates that the candidate artherosclerotic agent is efficacious.

26-27. (canceled)

28. A method for determining the therapeutic capacity of a candidate artherosclerotic agent, comprising:
providing a population of cells with a known PECAM-1 genotype status;
contacting the cells with a candidate composition, and
determining an effect of the candidate artherosclerotic agent on the subject, wherein a decrease in one or more of blood pressure, cholesterol level, blood glucose level, carbon monoxide levels, nitric oxide level, angina, heart attack, abnormal heart rhythms, heart failure, kidney failure, stroke, obstructed peripheral arteries, plaque rupture, tumor metastasis, tumor growth, lung function, cell aggregation, cell migration, total cholesterol (TC); triglyceride (TG); high density lipoprotein cholesterol (HDL-C); low density lipoprotein cholesterol (LDL-C); apolipoprotein A1 (apoA1); apolipoprotein B (apoB); lipoprotein(a) (Lp(a)), sP-selectin, PECAM-1, sPECAM-1, indicates that the candidate composition may be efficacious.

29-39. (canceled)

40. A nucleic acid array comprising wildtype and variant alleles of PECAM-1.

41. An isolated cell over-expressing a protein expressed from one or more of a homozygous wild type 125 allele; a homozygous 563 allele; heterozygous variant of the 125 allele; a homozygous variant of the 125 allele; a heterozygous variant of the 563 allele; a homozygous variant of the 563 allele; heterozygous variant of the 125 allele and a heterozygous variant of the 562 allele; heterozygous variant of the 125 allele and a homozygous variant of the 563 allele; a homozygous variant of the 125 allele and a homozygous variant of the 563 allele; a homozygous variant of the 125 allele and heterozygous variant of the 563 allele; or a homozygous wild type 125 allele;
a homozygous 563 allele of PECAM-1.

42. The isolated cell of claim 41, wherein the cell comprises one or more of PECAM-1CLeu, PECAM-1CSer or PECAM-1CLeu-PECAM-1CSer.

43-50. (canceled)

51. A kit for the assessment of artherosclerotic disease, comprising:
oligonucleotide probes that differentiate the wild-type and variant alleles of PECAM-1 and instructions for use, wherein the allele amino acid position 432, nucleotide position 373, amino acid position 563, or nucleotide position 1688 of PECAM-1.

52-54. (canceled)
